(19) United States
(12) Patent Application Publication
(10) Pub. No.: US 2005/0112574 A1
(43) Pub. Date: May 26, 2005

(54) Dna sequences for human angiogenesis genes

(76) Inventors: Jennifer Ruth Gamble, Stirling (AU); Christopher Norman Hahn, Modbury (AU); Mathew Alexander Vadas, Stirling (AU)

Correspondence Address: JENKINS, WILSON & TAYLOR, P. A., 3100 TOWER BLVD, SUITE 1400, DURHAM, NC, 27707, US

(21) Appl. No.: 10/489,740

(22) PCT Filed: Sep. 19, 2002
(86) PCT No.: PCT/AU02/01282

(30) Foreign Application Priority Data
Sep. 27, 2001 (AU) PR 7973
Sep. 27, 2001 (AU) PR 7974
Oct. 11, 2001 (AU) PR 8210
Oct. 29, 2001 (AU) PR 8532
Nov. 13, 2001 (AU) PR 8838
Aug. 28, 2002 (AU) 2002951032

(51) Int. Cl. C12Q 1/68; C07H 21/04; C12N 9/64

(52) U.S. Cl. 435/6; 435/69.1; 435/226; 435/320.1; 435/325; 536/23.2

(57) ABSTRACT

An isolated nucleic acid molecule comprising the sequence set forth in one of SEQ ID Numbers: 1 to 20.

### TECHNICAL FIELD

The present invention relates to novel nucleic acid sequences (“angiogenic genes”) involved in the process of angiogenesis. Each of the angiogenic genes encode a polypeptide that has a role in angiogenesis. In view of the realisation that these genes play a role in angiogenesis, the invention is also concerned with the therapy of pathologies associated with angiogenesis, the screening of drugs for pro- or anti-angiogenic activity, the diagnosis and prognosis of pathologies associated with angiogenesis, and in some cases the use of the nucleic acid sequences to identify and obtain full-length angiogenesis-related genes.

### BACKGROUND ART

The formation of new blood vessels from pre-existing vessels, a process termed angiogenesis, is essential for normal growth. Important angiogenic processes include those taking place in embryogenesis, renewal of the endometrium, formation and growth of the corpus luteum of pregnancy, wound healing and in the restoration of tissue structure and function after injury.

The formation of new capillaries requires a coordinated series of events mediated through the expression of multiple genes which may have either pro- or anti-angiogenic activities. The process begins with an angiogenic stimulus to existing vasculature, usually mediated by growth factors such as vascular endothelial growth factor or basic fibroblast growth factor. This is followed by degradation of the extracellular matrix, cell adhesion changes (and disruption), an increase in cell permeability, proliferation of endothelial cells (ECs) and migration of ECs towards the site of blood vessel formation. Subsequent processes include capillary tube or lumen formation, stabilisation and differentiation by the migrating ECs.

In the (normal) healthy adult, angiogenesis is virtually arrested and occurs only when needed. However, a number of pathological situations are characterised by enhanced, uncontrolled angiogenesis. These conditions include cancer, rheumatoid arthritis, diabetic retinopathy, psoriasis and cardiovascular diseases such as atherosclerosis. In other pathologies such as ischaemic limb disease or in coronary artery disease, growing new vessels through the promotion of an expanding vasculature would be of benefit.

A number of in vitro assays have been established which are thought to mimic angiogenesis and these have provided important tools to examine the mechanisms by which the angiogenic process takes place and the genes most likely to be involved.

Lumen formation is a key step in angiogenesis. The presence of vacuoles within ECs undergoing angiogenesis have been reported and their involvement in lumen formation has been postulated (Folkman and Haudenschild, 1980; Gamble et al., 1993). The general mechanism of lumen formation suggested by Folkman and Haudenschild (1980), has been that vacuoles form within the cytoplasm of a number of aligned ECs which are later converted to a tube. The union of adjacent tubes results in the formation of a continuous unicellular capillary lumen. However, little is known about the changes in cell morphology leading to lumen formation or the signals required for ECs to construct this feature.

An in vitro model of angiogenesis has been created from human umbilical vein ECs plated onto a 3 dimensional collagen matrix (Gamble et al., 1993). In the presence of phorbol myristate acetate (PMA) these cells form capillary tubes within 24 hours. With the addition of anti-integrin antibodies, the usually unicellular tubes (thought to reflect an immature, poorly differentiated phenotype) are converted to form a multicellular lumen through the inhibition of cell-matrix interactions and promotion of cell-cell interactions. This model has subsequently allowed the investigation of the morphological events which occur in lumen formation.

For the treatment of diseases associated with angiogenesis, understanding the molecular genetic mechanisms of the process is of paramount importance. The use of the in vitro model described above (Gamble et al., 1993), a model that reflects the critical events that occur during angiogenesis in vivo in a time dependent and broadly synchronous manner, has provided a tool for the identification of the key genes involved.

A number of genes have been identified from this model to be differentially expressed during the angiogenesis process. Functional analysis of a subset of these angiogenic genes and their effect on endothelial cell function and proliferation is described in detail below.

The isolation of these angiogenic genes has provided novel targets for the treatment of angiogenesis-related disorders.

### DISCLOSURE OF THE INVENTION

The present invention provides isolated nucleic acid molecules, which have been shown to be regulated in their expression during angiogenesis (see Tables 1 and 2).

In a first aspect of the present invention there is provided an isolated nucleic acid molecule as defined by SEQ ID Numbers: 1 to 20 and laid out in Table 1.

Following the realisation that these molecules, and those listed in Table 2, are regulated in their expression during angiogenesis, the invention provides isolated nucleic acid molecules as defined by SEQ ID Numbers: 1 to 114, and laid out in Tables 1 and 2, or fragments thereof, that play a role in an angiogenic process. Such a process may include, but is not restricted to, embryogenesis, menstrual cycle, wound repair, tumour angiogenesis and exercise induced muscle hypertrophy.

In addition, the present invention provides isolated nucleic acid molecules as defined by SEQ ID Numbers: 1 to 114, and laid out in Tables 1 and 2 (hereinafter referred to as “angiogenic genes”, “angiogenic nucleic acid molecules” or “angiogenic polypeptides” for the sake of convenience), or fragments thereof, that play a role in diseases associated with the angiogenic process. Diseases may include, but are not restricted to, cancer, rheumatoid arthritis, diabetic retinopathy, psoriasis, cardiovascular diseases such as atherosclerosis, ischaemic limb disease and coronary artery disease.

The invention also encompasses an isolated nucleic acid molecule that is at least 70% identical to any one of the angiogenic genes of the invention and which plays a role in the angiogenic process.

Such variants will have preferably at least about 85%, and most preferably at least about 95% sequence identity to the angiogenic genes. Any one of the polynucleotide variants described above can encode an amino acid sequence, which contains at least one functional or structural characteristic of the relevant angiogenic gene of the invention.

Sequence identity is typically calculated using the BLAST algorithm, described in Altschul et al (1997) with the BLOSUM62 default matrix.

The invention also encompasses an isolated nucleic acid molecule which hybridises under stringent conditions with any one of the angiogenic genes of the invention and which plays a role in an angiogenic process.

Hybridisation with PCR probes which are capable of detecting polynucleotide sequences, including genomic sequences, may be used to identify nucleic acid sequences which encode the relevant angiogenic gene. The specificity of the probe, whether it is made from a highly specific region, e.g., the 5′ regulatory region, or from a less specific region, e.g., a conserved motif, and the stringency of the hybridisation or amplification will determine whether the probe identifies only naturally occurring sequences encoding the angiogenic gene, allelic variants, or related sequences.

Probes may also be used for the detection of related sequences, and should preferably have at least 50% sequence identity to any of the angiogenic gene encoding sequences of the invention. The hybridisation probes of the subject invention may be DNA or RNA and may be derived from any one of the angiogenic gene sequences or from genomic sequences including promoters, enhancers, and introns of the angiogenic genes.

Means for producing specific hybridisation probes for DNAs encoding any one of the angiogenic genes include the cloning of polynucleotide sequences encoding the relevant angiogenic gene or its derivatives into vectors for the production of mRNA probes. Such vectors are known in the art, and are commercially available. Hybridisation probes may be labelled by radionuclides such as ³²P or ³⁵S, or by enzymatic labels, such as alkaline phosphatase coupled to the probe via avidin/biotin coupling systems, or other methods known in the art.

Under stringent conditions, hybridisation with ³²P labelled probes will most preferably occur at 42° C. in 750 mM NaCl, 75 mM trisodium citrate, 2% SDS, 50% formamide, 1× Denhart's, 10% (w/v) dextran sulphate and 100 μg/ml denatured salmon sperm DNA. Useful variations on these conditions will be readily apparent to those skilled in the art. The washing steps which follow hybridisation most preferably occur at 65° C. in 15 mM NaCl, 1.5 mM trisodium citrate, and 1% SDS. Additional variations on these conditions will be readily apparent to those skilled in the art.

The nucleotide sequences of the present invention can be engineered using methods accepted in the art so as to alter angiogenic gene-encoding sequences for a variety of purposes. These include, but are not limited to, modification of the cloning, processing, and/or expression of the gene product. PCR reassembly of gene fragments and the use of synthetic oligonucleotides allow the engineering of angiogenic gene nucleotide sequences. For example, oligonucleotide-mediated site-directed mutagenesis can introduce mutations that create new restriction sites, alter glycosylation patterns and produce splice variants etc.

As a result of the degeneracy of the genetic code, a number of polynucleotide sequences encoding the angiogenic genes of the invention, some that may have minimal similarity to the polynucleotide sequences of any known and naturally occurring gene, may be produced. Thus, the invention includes each and every possible variation of polynucleotide sequence that could be made by selecting combinations based on possible codon choices. These combinations are made in accordance with the standard triplet genetic code as applied to the polynucleotide sequence of the naturally occurring angiogenic gene, and all such variations are to be considered as being specifically disclosed.

The polynucleotides of this invention include RNA, cDNA, genomic DNA, synthetic forms, and mixed polymers, both sense and antisense strands, and may be chemically or biochemically modified, as will be appreciated by those skilled in the art. Such modifications include labels, methylation, intercalators, alkylators and modified linkages. In some instances it may be advantageous to produce nucleotide sequences encoding an angiogenic gene or its derivatives possessing a substantially different codon usage than that of the naturally occurring gene. For example, codons may be selected to increase the rate of expression of the peptide in a particular prokaryotic or eukaryotic host corresponding with the frequency that particular codons are utilized by the host. Other reasons to alter the nucleotide sequence encoding an angiogenic gene or its derivatives without altering the encoded amino acid sequence include the production of RNA transcripts having more desirable properties, such as a greater half-life, than transcripts produced from the naturally occurring sequence.

The invention also encompasses production of the nucleic acid molecules of the invention, entirely by synthetic chemistry. Synthetic sequences may be inserted into expression vectors and cell systems that contain the necessary elements for transcriptional and translational control of the inserted coding sequence in a suitable host. These elements may include regulatory sequences, promoters, 5′ and 3′ untranslated regions and specific initiation signals (such as an ATG initiation codon and Kozak consensus sequence) which allow more efficient translation of sequences encoding the angiogenic genes. In cases where the complete coding sequence including its initiation codon and upstream regulatory sequences are inserted into the appropriate expression vector, additional control signals may not be needed. However, in cases where only coding sequence, or a fragment thereof, is inserted, exogenous translational control signals as described above should be provided by the vector. Such signals may be of various origins, both natural and synthetic. The efficiency of expression may be enhanced by the inclusion of enhancers appropriate for the particular host cell system used (Scharf et al., 1994).

Nucleic acid molecules that are complements of the sequences described herein may also be prepared.

The present invention allows for the preparation of purified polypeptides or proteins. In order to do this, host cells may be transfected with a nucleic acid molecule as described above. Typically, said host cells are transfected with an expression vector comprising a nucleic acid molecule according to the invention. A variety of expression vector/host systems may be utilized to contain and express the sequences. These include, but are not limited to, microorganisms such as bacteria transformed with plasmid or cosmid DNA expression vectors; yeast transformed with yeast expression vectors; insect cell systems infected with viral expression vectors (e.g., baculovirus); or mouse or other animal or human tissue cell systems. Mammalian cells can also be used to express a protein that is encoded by a specific angiogenic gene of the invention using various expression vectors including plasmid, cosmid and viral systems such as a vaccinia virus expression system. The invention is not limited by the host cell employed.

The polynucleotide sequences, or variants thereof, of the present invention can be stably expressed in cell lines to allow long term production of recombinant proteins in mammalian systems. Sequences encoding any one of the angiogenic genes of the invention can be transformed into cell lines using expression vectors which may contain viral origins of replication and/or endogenous expression elements and a selectable marker gene on the same or on a separate vector. The selectable marker confers resistance to a selective agent, and its presence allows growth and recovery of cells which successfully express the introduced sequences. Resistant clones of stably transformed cells may be propagated using tissue culture techniques appropriate to the cell type.

The protein produced by a transformed cell may be secreted or retained intracellularly depending on the sequence and/or the vector used. As will be understood by those of skill in the art, expression vectors containing polynucleotides which encode a protein may be designed to contain signal sequences which direct secretion of the protein through a prokaryotic or eukaryotic cell membrane.

In addition, a host cell strain may be chosen for its ability to modulate expression of the inserted sequences or to process the expressed protein in the desired fashion. Such modifications of the polypeptide include, but are not limited to, acetylation, glycosylation, phosphorylation, and acylation. Post-translational cleavage of a “prepro” form of the protein may also be used to specify protein targeting, folding, and/or activity. Different host cells having specific cellular machinery and characteristic mechanisms for post-translational activities (e.g., CHO or HeLa cells), are available from the American Type Culture Collection (ATCC) and may be chosen to ensure the correct modification and processing of the foreign protein.

When large quantities of protein are needed such as for antibody production, vectors which direct high levels of expression may be used such as those containing the T5 or T7 inducible bacteriophage promoter. The present invention also includes the use of the expression systems described above in generating and isolating fusion proteins which contain important functional domains of the protein. These fusion proteins are used for binding, structural and functional studies as well as for the generation of appropriate antibodies.

In order to express and purify the protein as a fusion protein, the appropriate polynucleotide sequences of the present invention are inserted into a vector which contains a nucleotide sequence encoding another peptide (for example, glutathionine succinyl transferase). The fusion protein is expressed and recovered from prokaryotic or eukaryotic cells. The fusion protein can then be purified by affinity chromatography based upon the fusion vector sequence and the relevant protein can subsequently be obtained by enzymatic cleavage of the fusion protein.

Fragments of polypeptides of the present invention may also be produced by direct peptide synthesis using solid-phase techniques. Automated synthesis may be achieved by using the ABI 431A Peptide Synthesizer (Perkin-Elmer). Various fragments of polypeptide may be synthesized separately and then combined to produce the full length molecule.

In instances where the isolated nucleic acid molecules of the invention represent only partial gene sequence, these partial sequences can be used to obtain the corresponding sequence of the full-length angiogenic gene. Therefore, the present invention further provides the use of a partial nucleic acid molecule of the invention comprising a nucleotide sequence defined by any one of SEQ ID Numbers: 70, 72 to 73, 78, 83 to 87, 89, 160 or 174 to identify and/or obtain full-length human genes involved in the angiogenic process. Full-length angiogenic genes may be cloned using the partial nucleotide sequences of the invention by methods known per se to those skilled in the art. For example, in silico analysis of sequence databases such as those hosted at the National Centre for Biotechnology Information (http://www.ncbi.nlm.nih.gov/) can be searched in order to obtain overlapping nucleotide sequence. This provides a “walking” strategy towards obtaining the full-length gene sequence. Appropriate databases to search at this site include the expressed sequence tag (EST) database (database of GenBank, EMBL and DDBJ sequences from their EST divisions) or the non redundant (nr) database (contains all GenBank, EMBL, DDBJ and PDB sequences but does not include EST, STS, GSS, or phase 0, 1 or 2 HTGS sequences). Typically searches are performed using the BLAST algorithm described in Altschul et al (1997) with the BLOSUM62 default matrix. In instances where in silico “walking” approaches fail to retrieve the complete gene sequence, additional strategies may be employed. These include the use of “restriction-site PCR” will allows the retrieval of unknown sequence adjacent to a portion of DNA whose sequence is known. In this technique universal primers are used to retrieve unknown sequence. Inverse PCR may also be used, in which primers based on the known sequence are designed to amplify adjacent unknown sequences. These upstream sequences may include promoters and regulatory elements. In addition, various other PCR-based techniques may be used, for example a kit available from Clontech (Palo Alto, Calif.) allows for a walking PCR technique, the 5′RACE kit (Gibco-BRL) allows isolation of additional 5′ gene sequence, while additional 3′ sequence can be obtained using practised techniques (for eg see Gecz et al., 1997).

In a further aspect of the present invention there is provided an isolated polypeptide as defined by SEQ ID Numbers: 115 to 125 and laid out in Table 1.

The present invention also provides isolated polypeptides, which have been shown to be regulated in their expression during angiogenesis (see Tables 1 and 2).

More specifically, following the realisation that these polypeptides are regulated in their expression during angiogenesis, the invention provides isolated polypeptides as defined by SEQ ID Numbers: 115 to 216, and laid out in Tables 1 and 2, or fragments thereof, that play a role in an angiogenic process. Such a process may include, but is not restricted to, embryogenesis, menstrual cycle, wound repair, tumour angiogenesis and exercise induced muscle hypertrophy.

In addition, the present invention provides isolated polypeptides as defined by SEQ ID Numbers: 115 to 216, and laid out in Tables 1 and 2, or fragments thereof, that play a role in diseases associated with the angiogenic process. Diseases may include, but are not restricted to, cancer, rheumatoid arthritis, diabetic retinopathy, psoriasis, cardiovascular diseases such as atherosclerosis, ischaemic limb disease and coronary artery disease.

The invention also encompasses an isolated polypeptide having at least 70%, preferably 85%, and more preferably 95%, identity to any one of SEQ ID Numbers: 115 to 216, and which plays a role in an angiogenic process.

Sequence identity is typically calculated using the BLAST algorithm, described in Altschul et al (1997) with the BLOSUM62 default matrix.

In a further aspect of the invention there is provided a method of preparing a polypeptide as described above, comprising the steps of:
(1) culturing the host cells under conditions effective for production of the polypeptide; and
(2) harvesting the polypeptide.

According to still another aspect of the invention there is provided a polypeptide which is the product of the process described above.

Substantially purified protein or fragments thereof can then be used in further biochemical analyses to establish secondary and tertiary structure for example by x-ray crystallography of the protein or by nuclear magnetic resonance (NMR). Determination of structure allows for the rational design of pharmaceuticals to interact with the protein, alter protein charge configuration or charge interaction with other proteins, or to alter its function in the cell.

The invention has provided a number of genes likely to be involved in angiogenesis. As angiogenesis is critical in a number of pathological processes, the invention therefore enables therapeutic methods for the treatment of all angiogenesis-related disorders, and may enable the diagnosis or prognosis of all angiogenesis-related disorders associated with abnormalities in expression and/or function of any one of the angiogenic genes.

Examples of such disorders include, but are not limited to, cancer, rheumatoid arthritis, diabetic retinopathy, psoriasis, cardiovascular diseases such as atherosclerosis, ischaemic limb disease and coronary artery disease.

According to another aspect of the present invention there is provided a method of treating an angiogenesis-related disorder as described above, comprising administering a selective agonist or antagonist of an angiogenic gene or protein of the invention to a subject in need of such treatment.

Still further there is provided the use of a selective agonist or antagonist of an angiogenic gene or protein of the invention for the treatment of an angiogenesis-related disorder as described above.

For the treatment of angiogenesis-related disorders which result in uncontrolled or enhanced angiogenesis, including but not limited to, cancer, rheumatoid arthritis, diabetic retinopathy, psoriasis and cardiovascular diseases such as atherosclerosis, therapies which inhibit the expanding vasculature are desirable. This would involve inhibition of any one of the angiogenic genes or proteins that are able to promote angiogenesis, or enhancement, stimulation or re-activation of any one of the angiogenic genes or proteins that are able to inhibit angiogenesis.

For the treatment of angiogenesis-related disorders which are characterised by inhibited or decreased angiogenesis, including but not limited to, ischaemic limb disease and coronary artery disease, therapies which enhance or promote vascular expansion are desirable. This would involve inhibition of any one of the angiogenic genes or proteins that are able to restrict angiogenesis or enhancement, stimulation or re-activation of any one of the angiogenic genes or proteins that are able to promote angiogenesis.

For instance, antisense expression of BNO69 and BNO96 has been shown to inhibit endothelial cell growth and proliferation. Therefore, in the treatment of disorders where angiogenesis needs to be restricted, it would be desirable to inhibit the function of these genes. Alternatively, in the treatment of disorders where angiogenesis needs to be stimulated it may be desirable to enhance the function of these genes.

For each of these cases, the relevant therapy will be useful in treating angiogenesis-related disorders regardless of whether there is a lesion in the angiogenic gene.

Inhibiting Gene or Protein Function

Inhibiting the function of a gene or protein can be achieved in a variety of ways. Antisense nucleic acid methodologies represent one approach to inactivate genes whose altered expression is causative of a disorder. In one aspect of the invention an isolated nucleic acid molecule, which is the complement of any one of the relevant angiogenic nucleic acid molecules described above and which encodes an RNA molecule that hybridises with the mRNA encoded by the relevant angiogenic gene of the invention, may be administered to a subject in need of such treatment. Typically, a complement to any relevant one of the angiogenic genes is administered to a subject to treat or prevent an angiogenesis-related disorder.

In a further aspect of the invention there is provided the use of an isolated nucleic acid molecule which is the complement of any one of the relevant nucleic acid molecules of the invention and which encodes an RNA molecule that hybridises with the mRNA encoded by the relevant angiogenic gene of the invention, in the manufacture of a medicament for the treatment of an angiogenesis-related disorder.

Typically, a vector expressing the complement of a polynucleotide encoding any one of the relevant angiogenic genes may be administered to a subject to treat or prevent an angiogenesis-related disorder including, but not limited to, those described above. Many methods for introducing vectors into cells or tissues are available and equally suitable for use in vivo, in vitro, and ex vivo. For ex vivo therapy, vectors may be introduced into stem cells taken from the patient and clonally propagated for autologous transplant back into that same patient. Delivery by transfection, by liposome injections, or by polycationic amino polymers may be achieved using methods which are well known in the art. (For example, see Goldman et al., 1997).

Additional antisense or gene-targeted silencing strategies may include, but are not limited to, the use of antisense oligonucleotides, injection of antisense RNA, transfection of antisense RNA expression vectors, and the use of RNA interference (RNAi) or short interfering RNAs (siRNA). Still further, catalytic nucleic acid molecules such as DNAzymes and ribozymes may be used for gene silencing (Breaker and Joyce, 1994; Haseloff and Gerlach, 1988). These molecules function by cleaving their target mRNA molecule rather than merely binding to it as in traditional antisense approaches.

In a further aspect purified protein according to the invention may be used to produce antibodies which specifically bind any relevant angiogenic protein of the invention. These antibodies may be used directly as an antagonist or indirectly as a targeting or delivery mechanism for bringing a pharmaceutical agent to cells or tissues that express the relevant angiogenic protein. Such antibodies may include, but are not limited to, polyclonal, monoclonal, chimeric and single chain antibodies as would be understood by the person skilled in the art.

For the production of antibodies, various hosts including rabbits, rats, goats, mice, humans, and others may be immunized by injection with a protein of the invention or with any fragment or oligopeptide thereof, which has immunogenic properties. Various adjuvants may be used to increase immunological response and include, but are not limited to, Freund's, mineral gels such as aluminum hydroxide, and surface-active substances such as lysolecithin. Adjuvants used in humans include BCG (bacilli Calmette-Guerin) and Corynebacterium parvum.

It is preferred that the oligopeptides, peptides, or fragments used to induce antibodies to the relevant angiogenic protein have an amino acid sequence consisting of at least about 5 amino acids, and, more preferably, of at least about 10 amino acids. It is also preferable that these oligopeptides, peptides, or fragments are identical to a portion of the amino acid sequence of the natural protein and contain the entire amino acid sequence of a small, naturally occurring molecule. Short stretches of amino acids from these proteins may be fused with those of another protein, such as KLH, and antibodies to the chimeric molecule may be produced.

Monoclonal antibodies to any relevant angiogenic protein may be prepared using any technique which provides for the production of antibody molecules by continuous cell lines in culture. These include, but are not limited to, the hybridoma technique, the human B-cell hybridoma technique, and the EBV-hybridoma technique. (For example, see Kohler et al., 1975; Kozbor et al., 1985; Cote et al., 1983; Cole et al., 1984).

Monoclonal antibodies produced may include, but are not limited to, mouse-derived antibodies, humanised antibodies and fully human antibodies.

Antibodies may also be produced by inducing in vivo production in the lymphocyte population or by screening immunoglobulin libraries or panels of highly specific binding reagents as disclosed in the literature. (For example, see Orlandi et al., 1989; Winter et al., 1991).

Antibody fragments which contain specific binding sites for any relevant angiogenic protein may also be generated. For example, such fragments include, F(ab′)2 fragments produced by pepsin digestion of the antibody molecule and Fab fragments generated by reducing the disulfide bridges of the F(ab′)2 fragments. Alternatively, Fab expression libraries may be constructed to allow rapid and easy identification of monoclonal Fab fragments with the desired specificity. (For example, see Huse et al., 1989).

Various immunoassays may be used for screening to identify antibodies having the desired specificity. Numerous protocols for competitive binding or immunoradiometric assays using either polyclonal or monoclonal antibodies with established specificities are well known in the art. Such immunoassays typically involve the measurement of complex formation between a protein and its specific antibody. A two-site, monoclonal-based immunoassay utilizing monoclonal antibodies reactive to two non-interfering epitopes is preferred, but a competitive binding assay may also be employed.

In a further aspect, antagonists may include peptides, phosphopeptides or small organic or inorganic compounds. These antagonists should disrupt the function of any relevant angiogenic gene of the invention so as to provide the necessary therapeutic effect.

Peptides, phosphopeptides or small organic or inorganic compounds suitable for therapeutic applications may be identified using nucleic acids and polypeptides of the invention in drug screening applications as described below.

Enhancing Gene or Protein Function

Enhancing, stimulating or re-activating a gene's or protein's function can be achieved in a variety of ways. In one aspect of the invention administration of an isolated nucleic acid molecule, as described above, to a subject in need of such treatment may be initiated. Typically, any relevant angiogenic gene of the invention can be administered to a subject to treat or prevent an angiogenesis-related disorder.

In a further aspect, there is provided the use of an isolated nucleic acid molecule, as described above, in the manufacture of a medicament for the treatment of an angiogenesis-related disorder.

Typically, a vector capable of expressing any relevant angiogenic gene, or a fragment or derivative thereof, may be administered to a subject to treat or prevent a disorder including, but not limited to, those described above. Transducing retroviral vectors are often used for somatic cell gene therapy because of their high efficiency of infection and stable integration and expression. Any relevant full-length gene, or portions thereof, can be cloned into a retroviral vector and expression may be driven from its endogenous promoter or from the retroviral long terminal repeat or from a promoter specific for the target cell type of interest. Other viral vectors can be used and include, as is known in the art, adenoviruses, adeno-associated viruses, vaccinia viruses, papovaviruses, lentiviruses and retroviruses of avian, murine and human origin.

Gene therapy would be carried out according to established methods (Friedman, 1991; Culver, 1996). A vector containing a copy of any relevant angiogenic gene linked to expression control elements and capable of replicating inside the cells is prepared. Alternatively the vector may be replication deficient and may require helper cells for replication and use in gene therapy.

Gene transfer using non-viral methods of infection in vitro can also be used. These methods include direct injection of DNA, uptake of naked DNA in the presence of calcium phosphate, electroporation, protoplast fusion or liposome delivery. Gene transfer can also be achieved by delivery as a part of a human artificial chromosome or receptor-mediated gene transfer. This involves linking the DNA to a targeting molecule that will bind to specific cell-surface receptors to induce endocytosis and transfer of the DNA into mammalian cells. One such technique uses poly-L-lysine to link asialoglycoprotein to DNA. An adenovirus is also added to the complex to disrupt the lysosomes and thus allow the DNA to avoid degradation and move to the nucleus. Infusion of these particles intravenously has resulted in gene transfer into hepatocytes.

Although not identified to date, it is possible that certain individuals with angiogenesis-related disorders contain an abnormality in any one of the angiogenic genes of the invention. Therefore, in affected subjects that have decreased expression or activity of an angiogenic gene, a mechanism of down-regulation may be due to abnormal methylation of promoter regions of those angiogenic genes which contain CpG islands. Therefore in an alternative approach to therapy, administration of agents that remove abnormal promoter methylation may reactivate gene expression and restore normal function to the affected cell.

In affected subjects that express a mutated form of any one of the angiogenic genes of the invention it may be possible to prevent the disorder by introducing into the affected cells a wild-type copy of the gene such that it recombines with the mutant gene. This requires a double recombination event for the correction of the gene mutation. Vectors for the introduction of genes in these ways are known in the art, and any suitable vector may be used. Alternatively, introducing another copy of the gene bearing a second mutation in that gene may be employed so as to negate the original gene mutation and block any negative effect.

In a still further aspect, there is provided a method of treating an angiogenesis-related disorder comprising administering a polypeptide, as described above, or an agonist thereof, to a subject in need of such treatment.

In another aspect the invention provides the use of a polypeptide as described above, or an agonist thereof, in the manufacture of a medicament for the treatment of an angiogenesis-related disorder. Examples of such disorders are described above.

In a further aspect, a suitable agonist may also include peptides, phosphopeptides or small organic or inorganic compounds that can mimic the function of any relevant angiogenic gene, or may include an antibody to any relevant angiogenic gene that is able to restore function to a normal level.

Peptides, phosphopeptides or small organic or inorganic compounds suitable for therapeutic applications may be identified using nucleic acids and polypeptides of the invention in drug screening applications as described below.

In further embodiments, any of the agonists, antagonists, complementary sequences, nucleic acid molecules, proteins, antibodies, or vectors of the invention may be administered in combination with other appropriate therapeutic agents. Selection of the appropriate agents may be made by those skilled in the art, according to conventional pharmaceutical principles. The combination of therapeutic agents may act synergistically to effect the treatment or prevention of the various disorders described above. Using this approach, therapeutic efficacy with lower dosages of each agent may be possible, thus reducing the potential for adverse side effects.

Any of the therapeutic methods described above may be applied to any subject in need of such therapy, including, for example, mammals such as dogs, cats, cows, horses, rabbits, monkeys, and most preferably, humans.

Drug Screening

According to still another aspect of the invention, nucleic acid molecules of the invention as well as peptides of the invention, particularly any relevant purified angiogenic polypeptides or fragments thereof, and cells expressing these are useful for screening of candidate pharmaceutical compounds in a variety of techniques for the treatment of angiogenesis-related disorders.

Still further, it provides the use wherein high throughput screening techniques are employed.

Compounds that can be screened in accordance with the invention include, but are not limited to peptides (such as soluble peptides), phosphopeptides and small organic or inorganic molecules (such as natural product or synthetic chemical libraries and peptidomimetics).

In one embodiment, a screening assay may include a cell-based assay utilising eukaryotic or prokaryotic host cells that are stably transformed with recombinant nucleic acid molecules expressing the relevant angiogenic polypeptide or fragment, in competitive binding assays. Binding assays will measure for the formation of complexes between the relevant polypeptide or fragments thereof and the compound being tested, or will measure the degree to which a compound being tested will interfere with the formation of a complex between the relevant polypeptide or fragment thereof, and its interactor or ligand.

Non cell-based assays may also be used for identifying compounds that interrupt binding between the polypeptides of the invention and their interactors. Such assays are known in the art and include for example AlphaScreen technology (PerkinElmer Life Sciences, Mass., USA). This application relies on the use of beads such that each interaction partner is bound to a separate bead via an antibody. Interaction of each partner will bring the beads into proximity, such that laser excitation initiates a number of chemical reactions ultimately leading to fluorophores emitting a light signal. Candidate compounds that disrupt the binding of the relevant angiogenic polypeptide with its interactor will result in no light emission enabling identification and isolation of the responsible compound.

High-throughput drug screening techniques may also employ methods as described in WO84/03564. Small peptide test compounds synthesised on a solid substrate can be assayed through relevant angiogenic polypeptide binding and washing. The relevant bound angiogenic polypeptide is then detected by methods well known in the art. In a variation of this technique, purified angiogenic polypeptides can be coated directly onto plates to identify interacting test compounds.

An additional method for drug screening involves the use of host eukaryotic cell lines which carry mutations in any relevant angiogenic gene of the invention. The host cell lines are also defective at the polypeptide level. Other cell lines may be used where the gene expression of the relevant angiogenic gene can be regulated (i.e. over-expressed, under-expressed, or switched off). The host cell lines or cells are grown in the presence of various drug compounds and the rate of growth of the host cells is measured to determine if the compound is capable of regulating the growth of defective cells.

The angiogenic polypeptides of the present invention may also be used for screening compounds developed as a result of combinatorial library technology. This provides a way to test a large number of different substances for their ability to modulate activity of a polypeptide. The use of peptide libraries is preferred (see WO 97/02048) with such libraries and their use known in the art.

A substance identified as a modulator of polypeptide function may be peptide or non-peptide in nature. Non-peptide “small molecules” are often preferred for many in vivo pharmaceutical applications. In addition, a mimic or mimetic of the substance may be designed for pharmaceutical use. The design of mimetics based on a known pharmaceutically active compound (“lead” compound) is a common approach to the development of novel pharmaceuticals. This is often desirable where the original active compound is difficult or expensive to synthesise or where it provides an unsuitable method of administration. In the design of a mimetic, particular parts of the original active compound that are important in determining the target property are identified. These parts or residues constituting the active region of the compound are known as its pharmacophore. Once found, the pharmacophore structure is modelled according to its physical properties using data from a range of sources including x-ray diffraction data and NMR. A template molecule is then selected onto which chemical groups which mimic the pharmacophore can be added. The selection can be made such that the mimetic is easy to synthesise, is likely to be pharmacologically acceptable, does not degrade in vivo and retains the biological activity of the lead compound. Further optimisation or modification can be carried out to select one or more final mimetics useful for in vivo or clinical testing.

It is also possible to isolate a target-specific antibody and then solve its crystal structure. In principle, this approach yields a pharmacophore upon which subsequent drug design can be based as described above. It may be possible to avoid protein crystallography altogether by generating anti-idiotypic antibodies (anti-ids) to a functional, pharmacologically active antibody. As a mirror image of a mirror image, the binding site of the anti-ids would be expected to be an analogue of the original binding site. The anti-id could then be used to isolate peptides from chemically or biologically produced peptide banks.

Another alternative method for drug screening relies on structure-based rational drug design. Determination of the three dimensional structure of the polypeptides of the invention, or the three dimensional structure of the protein complexes which may incorporate these polypeptides allows for structure-based drug design to identify biologically active lead compounds.

Three dimensional structural models can be generated by a number of applications, some of which include experimental models such as x-ray crystallography and NMR and/or from in silico studies using information from structural databases such as the Protein Databank (PDB). In addition, three dimensional structural models can be determined using a number of known protein structure prediction techniques based on the primary sequences of the polypeptides (e.g. SYBYL—Tripos Associated, St. Louis, Mo.), de novo protein structure design programs (e.g. MODELER—MSI Inc., San Diego, Calif., or MOE—Chemical Computing Group, Montreal, Canada) or ab initio methods (e.g. see U.S. Pat. Nos. 5331573 and 5579250).

Once the three dimensional structure of a polypeptide or polypeptide complex has been determined, structure-based drug discovery techniques can be employed to design biologically-active compounds based on these three dimensional structures. Such techniques are known in the art and include examples such as DOCK (University of California, San Francisco) or AUTODOCK (Scripps Research Institute, La Jolla, Calif.). A computational docking protocol will identify the active site or sites that are deemed important for protein activity based on a predicted protein model. Molecular databases, such as the Available Chemicals Directory (ACD) are then screened for molecules that complement the protein model.

Using methods such as these, potential clinical drug candidates can be identified and computationally ranked in order to reduce the time and expense associated with typical ‘wet lab’ drug screening methodologies.

Compounds identified from the screening methods described above form a part of the present invention, as do pharmaceutical compositions containing these and a pharmaceutically acceptable carrier.

Pharmaceutical Preparations

Compounds identified from screening assays as indicated above can be administered to a patient at a therapeutically effective dose to treat or ameliorate a disorder associated with angiogenesis. A therapeutically effective dose refers to that amount of the compound sufficient to result in amelioration of symptoms of the disorder.

Toxicity and therapeutic efficacy of such compounds can be determined by standard pharmaceutical procedures in cell cultures or experimental animals. The data obtained from these studies can then be used in the formulation of a range of dosages for use in humans.

Pharmaceutical compositions for use in accordance with the present invention can be formulated in a conventional manner using one or more physiological acceptable carriers, excipients or stabilisers which are well known. Acceptable carriers, excipients or stabilizers are non-toxic at the dosages and concentrations employed, and include buffers such as phosphate, citrate, and other organic acids; antioxidants including absorbic acid; low molecular weight (less than about 10 residues) polypeptides; proteins, such as serum albumin, gelatin, or immunoglobulins; binding agents including hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or non-ionic surfactants such as Tween, Pluronics or polyethylene glycol (PEG).

The formulation of pharmaceutical compositions for use in accordance with the present invention will be based on the proposed route of administration. Routes of administration may include, but are not limited to, inhalation, insufflation (either through the mouth or nose), oral, buccal, rectal or parental administration.

Diagnostic and Prognostic Applications

Should abnormalities in any one of the angiogenic genes of the invention exist, which alter activity and/or expression of the gene to give rise to angiogenesis-related disorders, the polynucleotides and polypeptides of the invention may be used for the diagnosis or prognosis of these disorders, or a predisposition to such disorders. Examples of such disorders include, but are not limited to, cancer, rheumatoid arthritis, diabetic retinopathy, psoriasis, cardiovascular diseases such as atherosclerosis, ischaemic limb disease and coronary artery disease. Diagnosis or prognosis may be used to determine the severity, type or stage of the disease state in order to initiate an appropriate therapeutic intervention.

In another embodiment of the invention, the polynucleotides that may be used for diagnostic or prognostic purposes include oligonucleotide sequences, genomic DNA and complementary RNA and DNA molecules. The polynucleotides may be used to detect and quantitate gene expression in biopsied tissues in which abnormal expression or mutations in any one of the angiogenic genes may be correlated with disease. Genomic DNA used for the diagnosis or prognosis may be obtained from body cells, such as those present in the blood, tissue biopsy, surgical specimen, or autopsy material. The DNA may be isolated and used directly for detection of a specific sequence or may be amplified by the polymerase chain reaction (PCR) prior to analysis. Similarly, RNA or cDNA may also be used, with or without PCR amplification. To detect a specific nucleic acid sequence, direct nucleotide sequencing, reverse transcriptase PCR (RT-PCR), hybridisation using specific oligonucleotides, restriction enzyme digest and mapping, PCR mapping, RNAse protection, and various other methods may be employed. Oligonucleotides specific to particular sequences can be chemically synthesized and labelled radioactively or nonradioactively and hybridised to individual samples immobilized on membranes or other solid-supports or in solution. The presence, absence or excess expression of any one of the angiogenic genes may then be visualized using methods such as autoradiography, fluorometry, or colorimetry.

In a particular aspect, the nucleotide sequences of the invention may be useful in assays that detect the presence of associated disorders, particularly those mentioned previously. The nucleotide sequences may be labelled by standard methods and added to a fluid or tissue sample from a patient under conditions suitable for the formation of hybridisation complexes. After a suitable incubation period, the sample is washed and the signal is quantitated and compared with a standard value. If the amount of signal in the patient sample is significantly altered in comparison to a control sample then the presence of altered levels of nucleotide sequences in the sample indicates the presence of the associated disorder. Such assays may also be used to evaluate the efficacy of a particular therapeutic treatment regimen in animal studies, in clinical trials, or to monitor the treatment of an individual patient.

In order to provide a basis for the diagnosis or prognosis of an angiogenesis-related disorder associated with a mutation in any one of the angiogenic genes of the invention, the nucleotide sequence of the relevant gene can be compared between normal tissue and diseased tissue in order to establish whether the patient expresses a mutant gene.

In order to provide a basis for the diagnosis of a disorder associated with abnormal expression of any one of the angiogenic genes of the invention, a normal or standard profile for expression is established. This may be accomplished by combining body fluids or cell extracts taken from normal subjects, either animal or human, with a sequence, or a fragment thereof, encoding the relevant angiogenic gene, under conditions suitable for hybridisation or amplification. Standard hybridisation may be quantified by comparing the values obtained from normal subjects with values from an experiment in which a known amount of a substantially purified polynucleotide is used. Another method to identify a normal or standard profile for expression of any one of the angiogenic genes is through quantitative RT-PCR studies. RNA isolated from body cells of a normal individual, particularly RNA isolated from endothelial cells, is reverse transcribed and real-time PCR using oligonucleotides specific for the relevant gene is conducted to establish a normal level of expression of the gene. Standard values obtained in both these examples may be compared with values obtained from samples from patients who are symptomatic for a disorder. Deviation from standard values is used to establish the presence of a disorder.

Once the presence of a disorder is established and a treatment protocol is initiated, hybridisation assays or quantitative RT-PCR studies may be repeated on a regular basis to determine if the level of expression in the patient begins to approximate that which is observed in the normal subject. The results obtained from successive assays may be used to show the efficacy of treatment over a period ranging from several days to months.

According to a further aspect of the invention there is provided the use of an angiogenic polypeptide as described above in the diagnosis or prognosis of an angiogenesis-related disorder associated with any one of angiogenic genes of the invention, or a predisposition to such disorders.

When a diagnostic or prognostic assay is to be based upon any relevant angiogenic polypeptide, a variety of approaches are possible. For example, diagnosis or prognosis can be achieved by monitoring differences in the electrophoretic mobility of normal and mutant proteins. Such an approach will be particularly useful in identifying mutants in which charge substitutions are present, or in which insertions, deletions or substitutions have resulted in a significant change in the electrophoretic migration of the resultant protein. Alternatively, diagnosis or prognosis may be based upon differences in the proteolytic cleavage patterns of normal and mutant proteins, differences in molar ratios of the various amino acid residues, or by functional assays demonstrating altered function of the gene products.

In another aspect, antibodies that specifically bind the relevant angiogenic gene product may be used for the diagnosis or prognosis of disorders characterized by abnormal expression of the gene, or in assays to monitor patients being treated with the relevant angiogenic gene or protein or agonists, antagonists, or inhibitors thereof. Antibodies useful for diagnostic or prognostic purposes may be prepared in the same manner as described above for therapeutics. Diagnostic or prognostic assays may include methods that utilize the antibody and a label to detect the relevant protein in human body fluids or in extracts of cells or tissues. The antibodies may be used with or without modification, and may be labelled by covalent or non-covalent attachment of a reporter molecule.

A variety of protocols for measuring the relevant angiogenic polypeptide, including ELISAs, RIAS, and FACS, are known in the art and provide a basis for diagnosing altered or abnormal levels of expression. Normal or standard values for expression are established by combining body fluids or cell extracts taken from normal mammalian subjects, preferably human, with antibody to the relevant protein under conditions suitable for complex formation. The amount of standard complex formation may be quantitated by various methods, preferably by photometric means. Quantities of protein expressed in subject, control, and disease samples from biopsied tissues are compared with the standard values. Deviation between standard and subject values establishes the parameters for diagnosing disease.

Once an individual has been diagnosed or prognosed with a disorder, effective treatments can be initiated, as described above. In the treatment of angiogenesis-related diseases which are characterised by uncontrolled or enhanced angiogenesis, the expanding vasculature needs to be inhibited. This would involve inhibiting the relevant angiogenic genes or proteins of the invention that promote angiogenesis. In addition, treatment may also need to stimulate expression or function of the relevant angiogenic genes or proteins of the invention whose normal role is to inhibit angiogenesis but whose activity is reduced or absent in the affected individual.

In the treatment of angiogenesis-related diseases which are characterised by inhibited or decreased angiogenesis, approaches which enhance or promote vascular expansion are desirable. This may be achieved using methods essentially as described above but will involve stimulating the expression or function of the relevant angiogenic gene or protein whose normal role is to promote angiogenesis but whose activity is reduced or absent in the affected individual. Alternatively, inhibiting genes or proteins that restrict angiogenesis may also be an approach to treatment.

Microarray

In further embodiments, complete cDNAs, oligonucleotides or longer fragments derived from any of the polynucleotide sequences described herein may be used as probes in a microarray. The microarray can be used to monitor the expression level of large numbers of genes simultaneously and to identify genetic variants, mutations, and polymorphisms. This information may be used to determine gene function, to understand the genetic basis of a disorder, to diagnose or prognose a disorder, and to develop and monitor the activities of therapeutic agents. Microarrays may be prepared, used, and analysed using methods known in the art. (For example, see Schena et al., 1996; Heller et al., 1997).

Transformed Hosts

The present invention also provides for the production of genetically modified (knock-out, knock-in and transgenic), non-human animal models transformed with the nucleic acid molecules of the invention. These animals are useful for the study of the function of the relevant angiogenic gene, to study the mechanisms of disease as related to these genes, for the screening of candidate pharmaceutical compounds, for the creation of explanted mammalian cell cultures which express the protein or mutant protein and for the evaluation of potential therapeutic interventions.

Animal species which are suitable for use in the animal models of the present invention include, but are not limited to, rats, mice, hamsters, guinea pigs, rabbits, dogs, cats, goats, sheep, pigs, and non-human primates such as monkeys and chimpanzees. For initial studies, genetically modified mice and rats are highly desirable due to the relative ease in generating knock-in, knock-out or transgenics of these animals, their ease of maintenance and their shorter life spans. For certain studies, transgenic yeast or invertebrates may be suitable and preferred because they allow for rapid screening and provide for much easier handling. For longer term studies, non-human primates may be desired due to their similarity with humans.

To create an animal model based on any one of the angiogenic genes of the invention, several methods can be employed. These include generation of a specific mutation in a homologous animal gene, insertion of a wild type human gene and/or a humanized animal gene by homologous recombination, insertion of a mutant (single or multiple) human gene as genomic or minigene cDNA constructs using wild type or mutant or artificial promoter elements, or insertion of artificially modified fragments of the endogenous gene by homologous recombination. The modifications include insertion of mutant stop codons, the deletion of DNA sequences, or the inclusion of recombination elements (lox p sites) recognized by enzymes such as Cre recombinase.

To create transgenic mice in order to study gain of gene function in vivo, any relevant angiogenic gene can be inserted into a mouse germ line using standard techniques such as oocyte microinjection. Gain of gene function can mean the overexpression of a gene and its protein product, or the genetic complementation of a mutation of the gene under investigation. For oocyte injection, one or more copies of the wild type or mutant gene can be inserted into the pronucleus of a just-fertilized mouse oocyte. This oocyte is then reimplanted into a pseudo-pregnant foster mother. The liveborn mice can then be screened for integrants using analysis of tail DNA for the presence of the relevant human angiogenic gene sequence. The transgene can be either a complete genomic sequence injected as a YAC, BAC, PAC or other chromosome DNA fragment, a cDNA with either the natural promoter or a heterologous promoter, or a minigene containing all of the coding region and other elements found to be necessary for optimum expression.

To generate knock-out mice or knock-in mice, gene targeting through homologous recombination in mouse embryonic stem (ES) cells may be applied. Knock-out mice are generated to study loss of gene function in vivo while knock-in mice allow the study of gain of function or to study the effect of specific gene mutations. Knock-in mice are similar to transgenic mice however the integration site and copy number are defined in the former.

For knock-out mouse generation, gene targeting vectors can be designed such that they delete (knock-out) the protein coding sequence of the relevant angiogenic gene in the mouse genome. In contrast, knock-in mice can be produced whereby a gene targeting vector containing the relevant angiogenic gene can integrate into a defined genetic locus in the mouse genome. For both applications, homologous recombination is catalysed by specific DNA repair enzymes that recognise homologous DNA sequences and exchange them via double crossover.

Gene targeting vectors are usually introduced into ES cells using electroporation. ES cell integrants are then isolated via an antibiotic resistance gene present on the targeting vector and are subsequently genotyped to identify those ES cell clones in which the gene under investigation has integrated into the locus of interest. The appropriate ES cells are then transmitted through the germline to produce a novel mouse strain.

In instances where gene ablation results in early embryonic lethality, conditional gene targeting may be employed. This allows genes to be deleted in a temporally and spatially controlled fashion. As above, appropriate ES cells are transmitted through the germline to produce a novel mouse strain, however the actual deletion of the gene is performed in the adult mouse in a tissue specific or time controlled manner. Conditional gene targeting is most commonly achieved by use of the cre/lox system. The enzyme cre is able to recognise the 34 base pair loxP sequence such that loxP flanked (or floxed) DNA is recognised and excised by cre. Tissue specific cre expression in transgenic mice enables the generation of tissue specific knock-out mice by mating gene targeted floxed mice with cre transgenic mice. Knock-out can be conducted in every tissue (Schwenk et al., 1995) using the ‘deleter’ mouse or using transgenic mice with an inducible cre gene (such as those with tetracycline inducible cre genes), or knock-out can be tissue specific for example through the use of the CD19-cre mouse (Rickert et al., 1997).

According to still another aspect of the invention there is provided the use of genetically modified non-human animals for the screening of candidate pharmaceutical compounds.

It will be clearly understood that, although a number of prior art publications are referred to herein, this reference does not constitute an admission that any of these documents forms part of the common general knowledge in the art, in Australia or in any other country. Throughout this specification and the claims, the words “comprise”, “comprises” and “comprising” are used in a non-exclusive sense, except where the context requires otherwise.

### BRIEF DESCRIPTION OF THE DRAWINGS

FIG. 1. Examples of the classes of expression patterns of a number of angiogenic genes during angiogenesis as confirmed by Virtual Northern expression analysis. Each blot was probed with the control GAPDH1 gene to confirm loading of uniform cDNA amounts in blot construction between the defined time points of the assay.

FIG. 2. Detailed Virtual Northern expression analysis of the BNO69 gene. The top panels indicate the level of expression of BNO69 at varying time points in the in vitro model following stimulation of human umbilical vein endothelial cells (HUVECS) with phorbol myristate acetate (PMA) plus or minus (α2β1) antibody (AC11), vascular endothelial growth factor (VEGF), basic fibroblast growth factor (bFGF) or tumour necrosis factor (TNF). The lower panel shows expression levels of BNO69 in a number of human cell lines including K562 (erythroleukaemia), KG-1a (acute myelogenous leukaemia), Jurkat (acute T cell leukaemia), HeLa (cervical adenocarcinoma), HepG2 (liver tumour), LlM12-15 (colorectal carcinoma), MDA-MB-231 (breast cancer), DU145 (prostate cancer), HEK293 (embryonic kidney), HUSMC (primary umbilical vein smooth muscle cells) ±P (PMA). HUVEC T0 and HUVEC T3 represent HUVECs harvested from the 3-D model of angiogenesis at time 0 hours and 3 hours respectively.

FIG. 3. Detailed Virtual Northern expression analysis of the BNO96 gene. The top panels indicate the level of expression of BNO96 at varying time points in the in vitro model following stimulation of human umbilical vein endothelial cells (HUVECs) with phorbol myristate acetate (PMA) plus or minus (α2β1) antibody (AC11), vascular endothelial growth factor (VEGF), basic fibroblast growth factor (bFGF) or tumour necrosis factor (TNF). The lower panel shows expression levels of BNO69 in a number of human cell lines including K562 (erythroleukaemia), KG-1a (acute myelogenous leukaemia), Jurkat (acute T cell leukaemia), HeLa (cervical adenocarcinoma), HepG2 (liver tumour), LlM12-15 (colorectal carcinoma), MDA-MB-231 (breast cancer), DU145 (prostate cancer), HEK293 (embryonic kidney), HUSMC (primary umbilical vein smooth muscle cells) ±P (PMA). HUVEC T0 and HUVEC T3 represent HUVECs harvested from the 3-D model of angiogenesis at time 0 hours and 3 hours respectively.

FIG. 4. BNO69 in vitro regulation of human umbilical vein endothelial cell (HUVEC) function using retroviral-mediated gene transfer. The proliferation of HUVECs was measured over a 3 day period by direct cell counts. The mean ±SEM is given. Over-expression of antisense BNO69 (ASBNO69R) in HUVECs inhibits their proliferation. EV: Empty vector control.

FIG. 5. BNO69 in vitro regulation of human umbilical vein endothelial cell (HUVEC) function using adenoviral-mediated gene transfer. Over-expression of antisense BNO69 (ASBNO69A) in HUVECs leads to an inhibitory effect on cell proliferation.

FIG. 6. BNO69 in vitro regulation of human umbilical vein endothelial cell (HUVEC) function using retroviral-mediated gene transfer. Cell morphology of endothelial cells retrovirally transfected with either empty vector (EV) control or antisense BNO69 (ASBNO69R) is shown.

FIG. 7. Cell proliferation assay based on the over-expression of antisense BNO96 in human umbilical vein endothelial cells (HUVECs) using adenoviral-mediated gene transfer. Cells were infected with either vector only control (EV) or antisense BNO96 (ASBNO96), and harvested 48 hours later. Cell proliferation was measured by the colorimetric MTT assay performed 3 days after cell plating (mean ±SEM, n=4).

FIG. 8. Effect on cell migration as a result of over-expression of antisense BNO96 in human umbilical vein endothelial cells (HUVECs) using adenoviral-mediated gene transfer. Cells were infected with either vector only control (EV) or antisense BNO96 (ASBNO96) and migration of cells towards either no agent (Nil) or the chemotactic stimulant fibronectin (Fn) was measured after 18-24 hours.

FIG. 9. Effect on capillary tube formation on Matrigel as a result of over-expression of antisense BNO96 in human umbilical vein endothelial cells (HUVECs) using adenoviral-mediated gene transfer. Cells were infected with either vector only control (EV) or antisense BNO96 (ASBNO96) and assayed for tube formation over a 24 hour time period. Photos were taken after 20 hours. A and B: Low power photograph of tubes; C and D: High power photograph of tubes.

FIG. 10. Effect on capillary tube formation on collagen gels as a result of over-expression of antisense BNO96 in human umbilical vein endothelial cells (HUVECs) using adenoviral-mediated gene transfer. Cells were infected with either vector only control (EV) or antisense BNO96 (ASBNO96) and assayed for tube formation over an 18-24 hour time period. Photos were taken after 3 hours as the cells were migrating through the gel. M: Migrated cell. These appear flatter and less light refractive than non-migrated cells. NM: Non-migrated cell. These cells are rounded and light refractive.

FIG. 11. Effect on tumour necrosis factor (TNF)-induced E-selectin expression as a result of over-expression of antisense BNO96 in human umbilical vein endothelial cells (HUVECs) using adenoviral-mediated gene transfer. Cells were infected with either vector only control (EV) or antisense BNO96 (ASBNO96) and grown for 48 hours. TNF was added for 4 hours prior to staining for cell surface E-selectin expression using an anti E-selectin antibody. Detection was by phycoerythrin conjugated anti mouse antibody. The mean fluorescence intensity (MFI) is given.

cDNA synthesis protocol generated a majority of full length cDNAs which were subsequently PCR amplified for cDNA subtraction.

### EXAMPLE 3

### Suppression Subtractive Hybridisation (SSH)

SSH was performed on SMART amplified cDNA in order to enrich for cDNAs that were either up-regulated or down-regulated between the cDNA populations defined by the selected time-points. This technique also allowed “normalisation” of the regulated cDNAs, thereby making low abundance cDNAs (ie poorly expressed, but important, genes) more easily detectable. To do this, the PCR-Select cDNA synthesis kit (Clontech-user manual PT3041-1) and PCR-Select cDNA subtraction kit (Clontech-user manual PT1117-1) were used based on manufacturers conditions. These procedures relied on subtractive hybridisation and suppression PCR amplification. SSH was performed between the following populations: 0-0.5 hours; 0.5-3.0 hours; 3.0-6.0 hours; 6.0-24 hours.

### EXAMPLE 4

### Differential Screening of cDNA Clones

Following SSH, the cDNA fragments were digested with EagI and cloned into the compatible unique NotI site in pBluescript KS⁺ using standard techniques (Sambrook et al., 1989). This generated forward and reverse subtracted libraries for each time period. A differential screening approach outlined in the PCR-Select Differential Screening Kit (Clontech-user manual PT3138-1) was used to identify regulated cDNAs from non-regulated ones. To do this, cDNA arrays were generated by spotting clone plasmid DNA onto nylon filters in quadruplicate. Approximately 900 individual clones were analysed by cDNA array. These arrays were subsequently probed with:
a) unsubtracted time 1 cDNA (represents mRNAs present at time 1)
b) unsubtracted time 2 cDNA (represents mRNAs present at time 2)
c) forward subtracted cDNA (represents mRNAs upregulated at time 2)
d) reverse subtracted cDNA (represents mRNAs upregulated at time 1)

All hybridisations occurred at 42° C. in ExpressHyb solution (Clontech). Membranes were washed post-hybridisation according to kit instructions.

Those cDNA clones identified to be differentially expressed based on cDNA array hybridisations were subsequently sequenced. In silico database analysis was then used to identify homology to sequences present in the nucleotide and gene databases at the National Centre for Biotechnology Information (NCBI) in order to gain information about each clone that was sequenced. Selection of clones for further analysis was based upon the predicted function as deduced from homology searches.

Tables 1 and 2 provide information on the differentially expressed clones that were sequenced. Table 1 includes those clones which represent previously uncharacterised or novel genes, while Table 2 includes clones that correspond to previously identified genes which have not before been associated with angiogenesis. Also identified were a number of genes that have previously been shown to be involved in the process of angiogenesis. The identification of these clones provides a validation or proof of principle of the effectiveness of the angiogenic gene identification strategy employed and suggests that the clones listed in Tables 1 and 2 are additional angiogenic gene candidates.

An example from Table 1 is BNO69 which encodes a novel protein of 655 amino acids. Analysis of the full-length sequence of this clone indicated the presence of a GTPase Activating Protein (GAP) domain. GAP domains are found in a class of proteins that are key regulators of GTP binding proteins that include Ras, Rho, Cdc42 and Rac GTPases. These GTPases participate in many physiological processes which include cell motility, adhesion, cytokinesis, proliferation, differentiation and apoptosis (reviewed in Van Aelst and D'Souza-Schorey, 1997; Ridley, 2001). Rho-like GTPAses cycle between an inactive GDP bound state and an active GTP bound state. The conversion between the two forms is regulated primarily by two types of proteins. They are up-regulated by the guanine exchange factors (GEFs), which enhance the exchange of bound GDP for GTP, and down-regulated by the GTPase-activating proteins (GAPS) which increase the intrinsic rate of hydrolysis of bound GTP. When loaded with GTP, Rho GTPases gain the ability to bind a set of downstream effectors, leading for example to various cytoskeletal rearrangements.

An example from Table 2 is the BNO96 gene. Sequencing of cDNA clone 23 (BNO96) established that this clone was identical to the gammal2 subunit (GNG12) of the G protein. Heterotrimeric G proteins are involved in signal transduction from cell surface receptors to cellular effectors. The G proteins are composed of alpha (α), beta (β) and gamma (γ) subunits. Upon stimulation the α subunit dissociates from the complex and both the α and the βγ subunits are able to activate multiple effectors to generate many intracellular signals.

At present 6 different β and 12 different γ subunits have been identified. Since the γγ subunits are tightly associated and form highly stable dimers, they have been considered as a functional unit to date.

GNG12 has been reported to be widely expressed and rich in fibroblasts and smooth muscle cells (Ueda et al., 1999). GNG12 is a substrate for protein kinase C and is phosphorylated following stimulation with agents such as PMA, LPA (lysophosphatidic acid), growth factors and serum (Asano et al., 1998). GNG12 is also associated with F-actin (Ueda et al., 1997).

Previous reports have shown that over-expression of GNG12 alone has no effect on NIH-3T3 fibroblasts. However, over-expression of the β₁γ₁₂ dimer induced cell rounding, disruption of stress fibres and enhancement of cell migration. Phosphorylation of GNG12 is required for its effects on cell motility (Yasuda et al., 1998).

Based on available information regarding the BNO69 and BNO96 genes, and given that both genes have been shown to be differentially expressed during angiogenesis in the present invention, there is the suggestion that the these genes have features consistent with them performing functions associated with angiogenesis and for this reason they were analysed further.

### EXAMPLE 5

### Virtual Northern Blot Analysis

Before functional analysis of selected clones, the differential expression observed from the cDNA array analysis of the clones listed in Tables 1 and 2 (including BNO69 and BNO96) was confirmed by Virtual Northern analysis.

Amplified cDNA from each time point was electrophoresed on an agarose/EtBr gel and the cDNA was transferred to a nylon membrane using Southern transfer according to established techniques (Sambrook et al., 1989). All cDNA clone inserts were labelled with ³²P using the Megaprime DNA labelling system (Amersham Pharmacia Biotech) and hybridisations were performed in ExpressHyb solution (Clontech) according to manufacturers specifications.

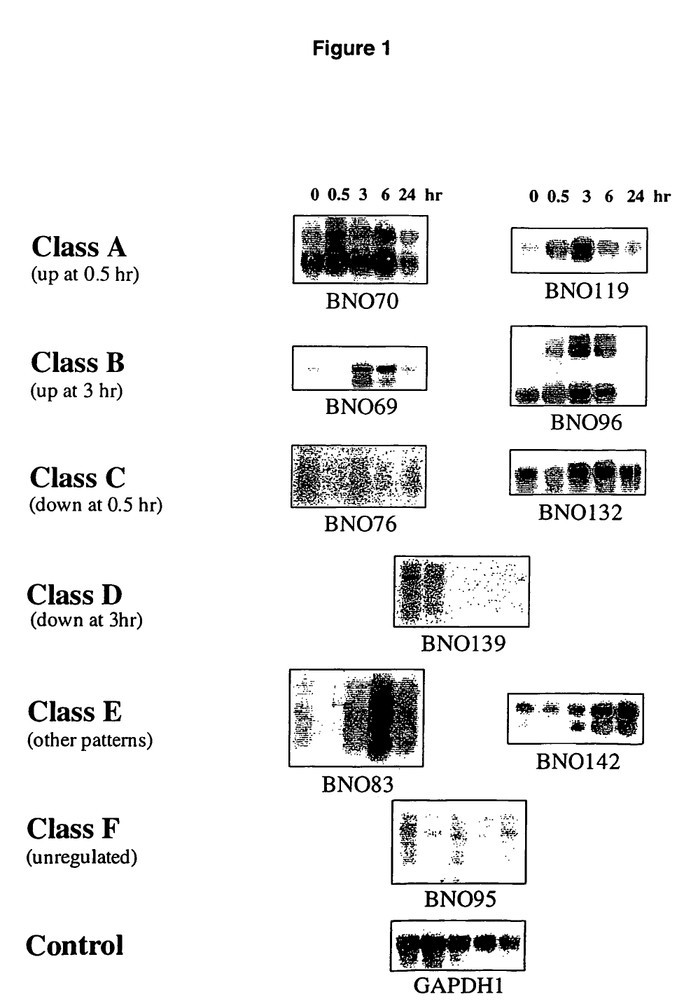

Based on the results, clones were grouped according to their type of regulation pattern (FIG. 1, and Tables 1 and 2). Of the 20 novel genes identified to date, 9 were confirmed to be regulated during angiogenesis, 4 gave an undetectable signal on Virtual Northern blots and the remaining clones did not indicate regulation of expression based on the Virtual Northern result. Similarly, of the 94 known genes not previously associated with angiogenesis, 59 were confirmed to be differentially regulated from the angiogenesis model. Those clones that did not display differential expression (Class F) or did not give detectable results on Virtual Northerns may still be involved in angiogenesis however further characterisation is needed.

### EXAMPLE 6

### Cell and Stimulation Specificity

To further characterise the differentially expressed clones and confirm their role in angiogenesis, virtual Northern blots were again used to determine the cell type expression specificity and their stimulation in monolayer cultures with specific growth factors. Endothelial cells were plated on a 2-dimensional (2-D) collagen matrix and were stimulated for 0.5, 3.0, 6.0 and 24 hours with vascular endothelial growth factor (VEGF), basic fibroblast growth factor (FGF), tumour necrosis factor a (TNFα), PMA+ACII, or PMA, alone. Primary cultures of endothelial cells, fibroblasts, smooth muscle cells, together with tumour cell lines were collected. RNA was prepared from all cells and the SMART-PCR cDNA synthesis kit (Clontech-user manual PT3041-1) was used to generate cDNA for virtual Northern preparation. Prepared blots were then probed for regulation of the specific angiogenic gene of interest. Results are shown in Tables 1 and 2.

Of the clones so far analysed, all were confirmed to be expressed in endothelial cells. Of those clones listed in Table 1, three of the six clones analysed for signal specificity were shown to be influenced by the presence of VEGF, FGF and PMA. Two clones showed no response following the stimulation of endothelial cells in culture and the remaining clone showed that the differential expression was specific for the 3-dimensional and not 2-dimensional collagen gels. Of those clones listed in Table 2, two of the six clones analysed for signal specificity were shown to be influenced by the presence of VEGF and FGF and one clone was influenced by the presence of PMA only. One clone showed no response following the stimulation of endothelial cells in culture and the remaining two clones showed that the differential expression was specific for the 3-dimensional and not 2-dimensional collagen gels.

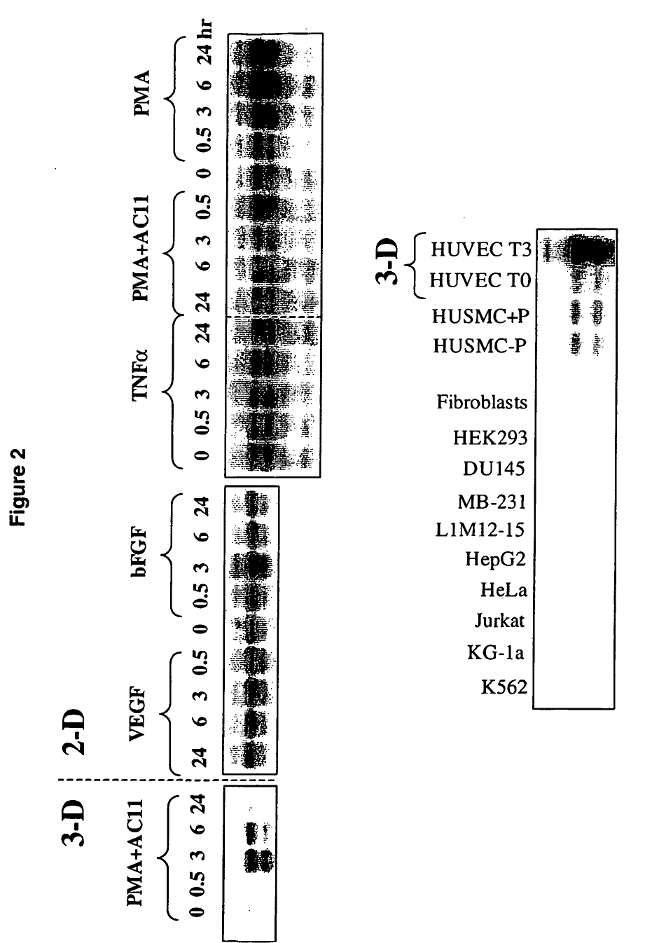

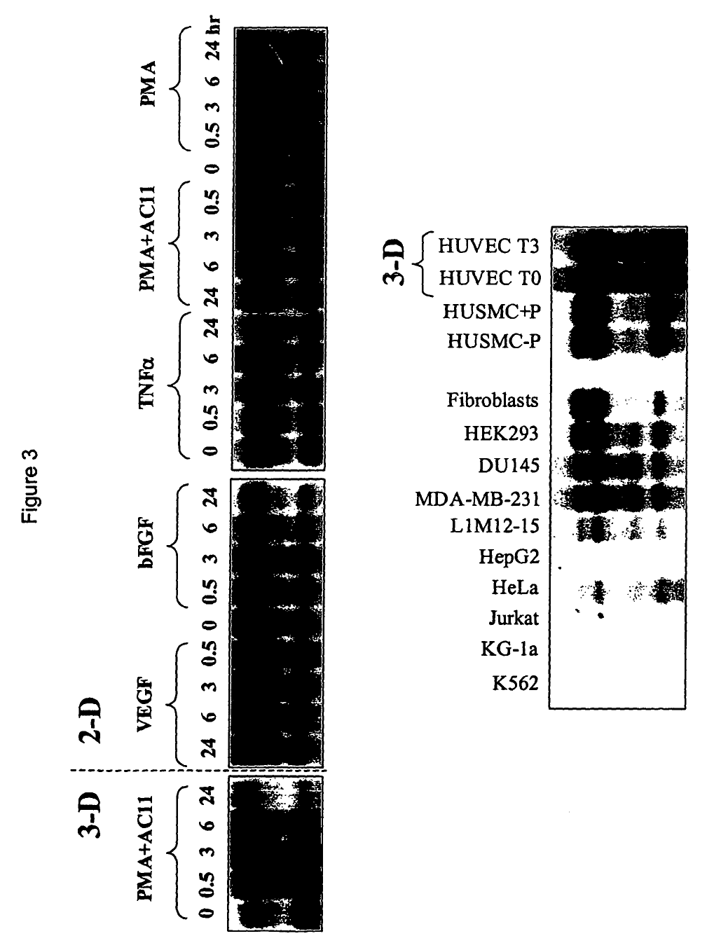

FIGS. 2 and 3 provide a detailed summary of the cell and stimulation specificity results for the BN069 and BNo96 genes respectively. These results indicate that both genes are up-regulated at the 3-hour time-point of the 3-dimensional (3-D) in vitro model. While the BN069 gene is expressed in response to FGF, VEGF and PMA, expression of the BN096 gene occurs only in response to PMA. Both genes are expressed in several cell types including endothelial cells.

### EXAMPLE 7

### Analysis of the Angiogenic Genes

The genes identified by this study to be implicated in the angiogenesis process, as listed in Tables 1 and 2, may be used for further studies in order to confirm their role in angiogenesis in vitro. To do this, full-length coding sequences of the genes can be cloned into suitable expression vectors such as retroviruses or adenoviruses in both sense and anti-sense orientations and used for infection into endothelial cells (ECs). Retrovirus infection gives long-term EC lines expressing the gene of interest whereas adenovirus infection gives transient gene expression. Infected cells can then be subjected to a number of EC assays including proliferation and capillary tube formation to confirm the role of each gene in angiogenesis.

As an example, the effect of BN069 and BN096 on in vitro regulation of EC function has been determined and is described below.

In Vitro Regulation of EC Function—BN069

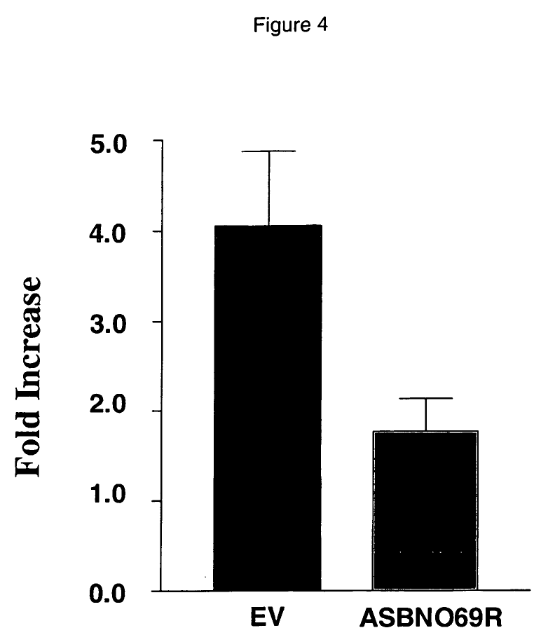

The effect of BN069 on endothelial cell function and angiogenesis involved transfection of the antisense of BN069 into endothelial cells by retroviral or adenoviral mediated gene transfer. Human umbilical vein endothelial cells (HUVECs) at passage 1 or 2 were used for the overexpression experiments. Initially, the BNO69 gene was cloned into the replication defective retrovirus pRufNeo (Rayner and Gonda, 1994). The commercially available cell line BING was used for transfection and production of viral supernatant. HUVEC clones infected with the retrovirus and expressing the antisense BNO69 gene were selected for neo resistance using G418 and pooled together for further growth and analysis. The proliferation of the pooled clones was measured over a 3 day period by direct cell counts. Results of these experiments indicated that cells that had been infected with the antisense construct of BNO69 showed a decrease in their proliferative potential (FIG. 4).

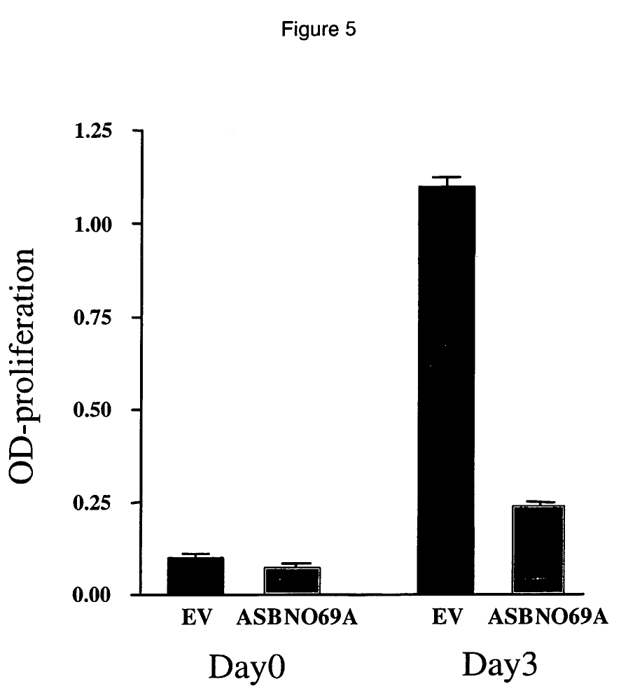

Subsequent experiments using adenoviral-mediated expression of antisense BNO69 in HUVECs showed a similar effect on cell proliferation as that observed in the retroviral system. HUVECs were infected with either vector only control or antisense BNO69 and were harvested 24 hours after infection and plated onto microtitre plates in complete growth medium. Cell proliferation was measured by the colorimetric MTT assay as described previously (Xia et al., 1999). The assay was performed 3 days after cell plating. Results of these experiments showed that the proliferation of HCs was inhibited by adenoviral-mediated expression of antisense BNO69 (FIG. 5).

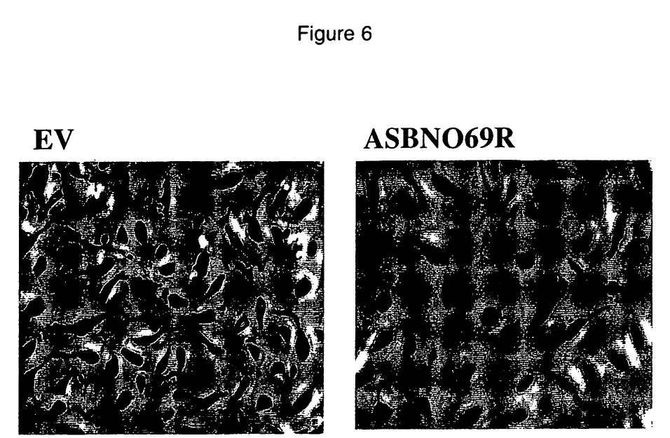

In addition, in both the retrovirus and adenovirus infection systems, a major feature of the cells infected with the antisense construct to BNO69 was the change in cell morphology. Cells appeared enlarged in size, with an increase in the extent of the cytoplasm (FIG. 6). The increase in cell size was confirmed by analysis on a fluorescence activated cell sorter where a measurement of both the forward scatter and side scatter gives information on the size and granularity of the cells respectively. In both retrovirus and adenovirus systems these parameters were changed. In retrovirus infected cells, forward scatter was 385 (EV) and 522 (ASBNO69R) while side scatter measured 289 (EV) and 508 (ASBNO69R). In the adenovirus infected cells the measurements for forward scatter were, 444 (EV) and 533 (ASBNO69R) while side scatter measured 417 (EV) and 500 (ASBNO69R).

In Vitro Regulation of EC Function—BNO96

The effect of BNO96 on endothelial cell function and angiogenesis involved transfection of the antisense of BNO96 into endothelial cells by adenoviral gene transfer. Initially, antisense BNO96 was produced as a recombinant adenoviral plasmid employing homologous recombination in bacteria (essentially as outlined in http://coloncancer.org/protocol.htm). The resultant plasmids were transfected into the mammalian packaging cell line 293 for expansion of virus, and the virus was subsequently purified by caesium chloride gradients. Transfection efficiency was assessed by green fluorescent protein and plaque forming units as given in the protocol above.

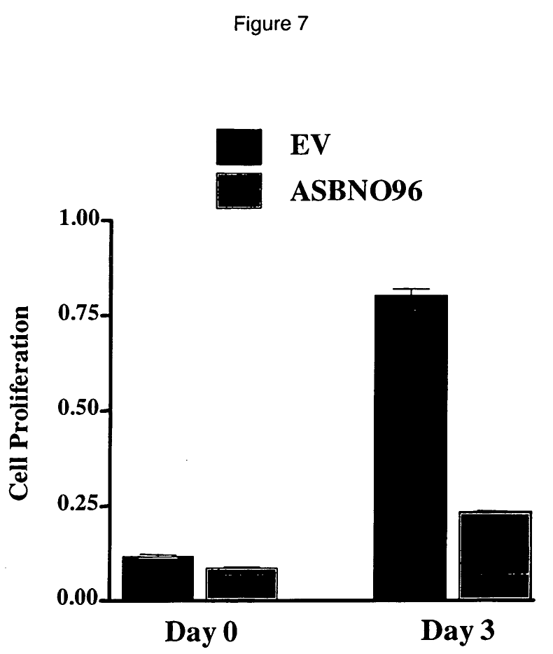

Initially, the effect on endothelial cell proliferation of antisense BNO96 was determined. Human umbilical vein endothelial cells (HUVECs) were infected with either vector only control or antisense BNO96 and were harvested 48 hours later. Cell proliferation was measured by the colorimetric MTT assay as described previously (Xia et al., 1999). The assay was performed 3 days after cell plating (mean ±SEM, n=4). Infection of HUVECs with antisense BNO96 was found to inhibit cell proliferation (FIG. 7) when cells were cultured in full growth medium.

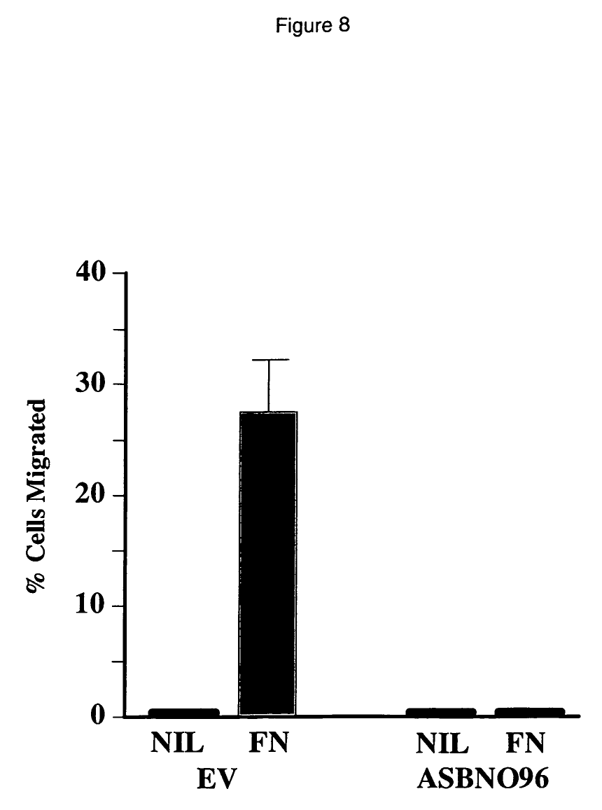

Another feature of the angiogenic in vitro model is the migration of endothelial cells into the matrix. To test the effect that BNO96 plays on this process, cell migration experiments were next conducted. Human umbilical vein endothelial cells (HUVECs) were infected with either vector only control or antisense BNO96 and migration of cells towards either no agent or the chemotactic stimulant fibronectin was measured. The migration assay was performed as previously described (Leavesley et al., 1993). Briefly, fibronectin at 50 μg/ml was coated on the under-side of 8.0 μm Transwell filters to act as a chemotactic gradient. Cell migration was assessed after 18-24 hours. Results from these experiments showed that antisense BNO96-infected cells were inhibited from migrating towards fibronectin as a chemotactic stimulant (FIG. 8).

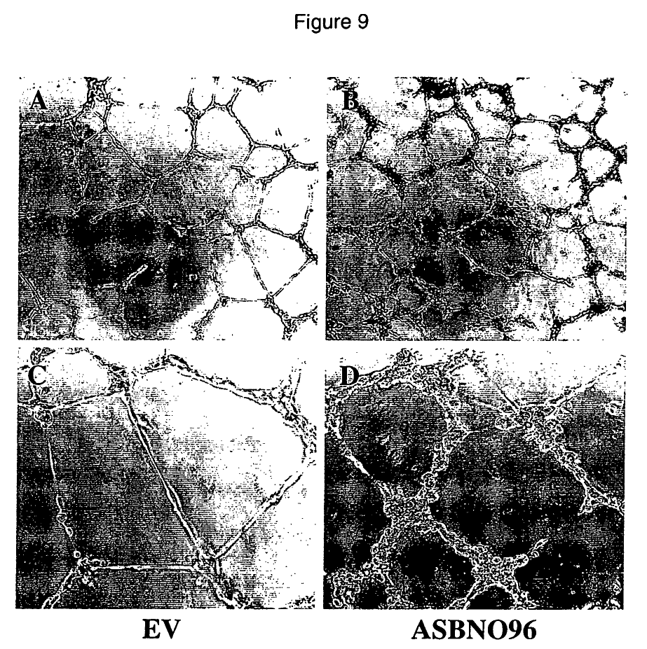

An essential feature of the angiogenic process is the formation of capillary tubes. The role that BNO96 plays in this process was measured using the Matrigel and collagen gel models. In the Matrigel system, human umbilical vein endothelial cells (HUVECs) were infected with either vector only control or antisense BNO96 and assayed for tube formation as previously described (Cockerill et al., 1994). Briefly, 140 μl of 3×10⁵ cells/ml were plated onto the Matrigel and cell reorganisation and tube formation was assessed over a 24 hour time period. The antisense BNO96-infected cells failed to make capillary tubes in the Matrigel capillary tube assay (FIG. 9).

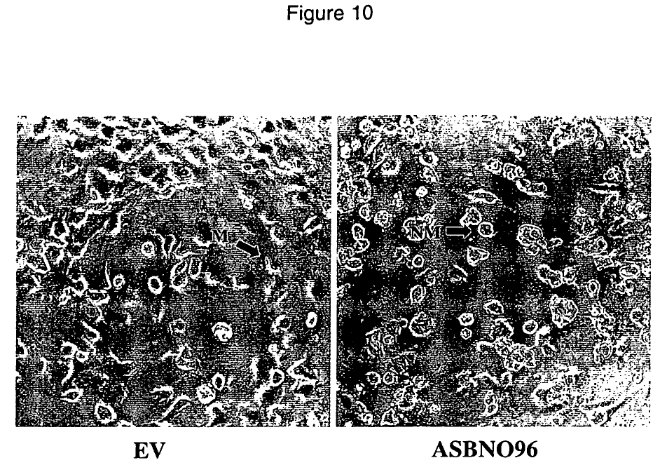

In the collagen gel model, HUVECs were again infected with either vector only control or antisense BNO96 and assayed over an 18-24 hour time period for tube formation as previously described (Gamble et al., 1993). Expression of antisense BNO96 resulted in inhibition of cell migration (and subsequent tube formation) into the collagen gel (FIG. 10).

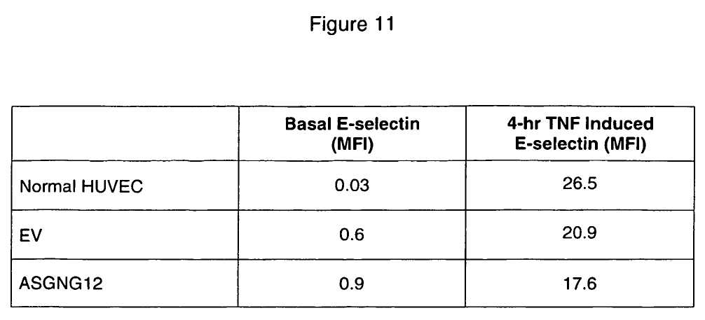

The next experiment addressed the question of whether the inhibition of BNO96 produces endothelial cell changes that are specific for functions associated with angiogenesis. E-selectin is an endothelial specific adhesion molecule that is induced by inflammatory cytokines such as TNF and IL-1 and mediates neutrophil-endothelial cell interactions. The effect on E-selectin expression as a result of over-expression of antisense BNO96 in human umbilical vein endothelial cells (HUVECs) was therefore determined. Methods used were as described in Litwin et al (1997). Briefly, HUVECs were infected with either vector only control or antisense BNO96 and grown for 48 hours. Following this, cells were transferred to 24-well trays and incubated overnight. Tumour necrosis factor (TNF) at 0.5 ng/ml was added for 4 hours prior to staining for cell surface E-selectin expression using an anti E-selectin antibody. Detection was by phycoerythrin conjugated anti mouse antibody. The result of these experiments showed that cells over-expressing the antisense BNO96 gene still responded in a normal fashion to the pro-inflammatory stimulant, tumour necrosis factor, to induce the adhesion molecule E-selectin (FIG. 11). This suggests that the effect of antisense BNO96 on endothelial cell function is selective.

The capacity of antisense BNO96 to inhibit cell proliferation, migration and capillary tube formation but not TNF induced E-selectin expression may suggest that knockdown of the BNO96 gene specifically affects the angiogenic capacity of endothelial cells. Other cell functions such as their ability to participate in inflammatory reactions would appear to be normal (as far as those measured to date). The BNO96 gene may therefore play a defining role in the angiogenesis process and is a target for the development of therapeutics for the treatment of angiogenesis-related pathologies.

Protein Interaction Studies

The ability of any one of the angiogenic proteins of the invention, including BNO69 and BNO96, to bind known and unknown proteins can be examined. Procedures such as the yeast two-hybrid system are used to discover and identify any functional partners. The principle behind the yeast two-hybrid procedure is that many eukaryotic transcriptional activators, including those in yeast, consist of two discrete modular domains. The first is a DNA-binding domain that binds to a specific promoter sequence and the second is an activation domain that directs the RNA polymerase II complex to transcribe the gene downstream of the DNA binding site. Both domains are required for transcriptional activation as neither domain can activate transcription on its own. In the yeast two-hybrid procedure, the gene of interest or parts thereof (BAIT), is cloned in such a way that it is expressed as a fusion to a peptide that has a DNA binding domain. A second gene, or number of genes, such as those from a cDNA library (TARGET), is cloned so that it is expressed as a fusion to an activation domain. Interaction of the protein of interest with its binding partner brings the DNA-binding peptide together with the activation domain and initiates transcription of the reporter genes. The first reporter gene will select for yeast cells that contain interacting proteins (this reporter is usually a nutritional gene required for growth on selective media). The second reporter is used for confirmation and while being expressed in response to interacting proteins it is usually not required for growth.

The nature of the interacting genes and proteins can also be studied such that these partners can also be targets for drug discovery.

Structural Studies

Recombinant angiogenic proteins of the invention can be produced in bacterial, yeast, insect and/or mammalian cells and used in crystallographical and NMR studies. Together with molecular modeling of the protein, structure-driven drug design can be facilitated.

TABLE 1
Novel Angiogenesis Genes

| | Time Course | Signal | Cell Type | Virtual | | UniGene Cluster | SEQ ID |
|---|---|---|---|---|---|---|---|
| Gene | Subtraction¹ | Specificity² | Specificity³ | Northern⁴ | Homology Details | Number | Numbers |
| BNO69 | 0.5-3 | V/F/P | S | B | Hypothetical GAP domain containing protein | Hs 93589 | 1, 115 |
| BNO71 | 3.0-6.0 | V/F/P | M | B | LUZP—leucine zipper protein. Putative | Hs 334673 | 2, 116 |
| | | | | | transcription factor | | |
| BNO72 | 0.5-3.0 | 3-D v,weak | | B | No EST matches | None | 3 |
| BNO73 | 0.5-3.0 | V/F/P | | B | ESTs | Hs 315562 | 4 |
| BNO77 | 0.5-3.0 | NR | | C | Archease-like protein | Hs 292812 | 5, 117 |
| BNO79 | 0.5-0 | | | E? | CRELD1 (Cysteine-rich with EGF-like | Hs 9383 | 6, 118 |
| | | | | | domains 1) | | |
| BNO82 | 0.5-3.0 | NR | M | E? | Hypothetical protein | Hs 172069 | 7, 119 |
| BNO83 | 3.0-6.0 | | | E (↑6 hr) | No EST matches | None | 8 |
| BNO84 | 3.0-6.0 | | | E (↓6 hr) | EST | None | 9 |
| BNO85 | 0-0.5 | | | undetectable | No EST matches | None | 10 |
| BNO86 | 0.5-3.0 | | | undetectable | EST | None | 11 |
| BNO87 | 0-0.5 | | | undetectable | No EST matches | None | 12 |
| BNO88 | 0-0.5 | | | undetectable | Hypothetical protein | Hs 4863 | 13, 120 |
| BNO89 | 0.5-3.0 | | | F | No EST matches | None | 14 |
| BNO90 | 0-0.5 | | | F | ESTs | Hs 28893 | 15, 121 |
| BNO92 | 3.0-6.0 | | | F | HMGE(GrpE-like protein cochaperone) | Hs 151903 | 16, 122 |
| BNO94 | 0-0.5 | | | F | KIAA0678 | Hs 12707 | 17, 123 |
| BNO95 | 0-0.5 | | | F | Hypothetical protein | Hs 17283 | 18, 124 |
| BNO160 | 0-0.5 | | | | EST, Moderately similar to GA15_HUMAN | Hs 335776 | 19 |
| | | | | | Growth arrest and DNA-damage-inducible protein | | |
| | | | | | GADD153 (DNA-damage inducible transcript 3) | | |
| | | | | | (DDIT3) (C/EBP-homologous protein) (CHOP) | | |
| BNO174 | 0-0.5 | | | L | KIAA0251 | Hs 343566 | 20, 125 |

Note:
¹The time period in which isolated clones were obtained and the direction of subtraction;
²Response to VEGF (V), bFGF (F), PMA (P) on 2-dimensional collagen gels. Response specific to 3-dimensional (3-D) not 2-dimensional collagen gels. No response (NR);
³Expression in many (M) or several (S) cell types.
⁴Specifically relates to the tube and lumen forming assay on 3-dimensional collagen. Class B: up at 3 hours; Class C: down at 0.5 hours; Class E: other regulation patterns; Class F: not regulated; Class L: Virtual Northern limited to 0 and 0.5 hour time points only, but no regulation.

TABLE 2
Genes with a Previously Unknown Role in Angiogenesis

| | | | | | | UniGene | |
|---|---|---|---|---|---|---|---|
| | Time Course | Signal | Cell Type | Virtual | | Cluster | SEQ ID |
| BNO # | Subtraction¹ | Specificity² | Specificity³ | Northern⁴ | Homology Details | Number | Numbers |
| BNO65 | 0-0.5 | | | A | SDF-2 (Stromal cell-derived factor 2) | Hs 118684 | 21, 126 |
| BNO66 | 0-0.5 | | | A | PRO1992 (Similar to arginyl-tRNA synthetase) | Hs 15395 | 22, 127 |
| BNO67 | 0.5-0 | | | A | Putative protein (Thioredoxin related protein) | Hs 6101 | 23, 128 |
| BNO70 | 0-0.5 | V/F | | B | BRG1-binding protein ELD/OSA1 | Hs 73287 | 24, 129 |
| BNO74 | 3.0-6.0 | | | B | SET domain-containing protein 7 | Hs 78521 | 25, 130 |
| BNO75 | 3.0-6.0 | | | B? (weak) | VPS35 (Vacuolar protein sorting 35) | Hs 264190 | 26, 131 |
| BNO76 | 3.0-6.0 | | | C | EBRP (Emopamil binding related protein, | Hs 298490 | 27, 132 |
| | | | | | delta8-delta7 sterol isomerase related protein) | | |
| BNO78 | 0-0.5 | | | E (↓24 hr) | CPSF2 (Cleavage and polyadenylation | Hs 224961 | 28, 133 |
| | | | | | specific factor 2) | | |
| BNO80 | 0.5-0 | | | E | Hypothetical protein | Hs 323193 | 29, 134 |
| BNO91 | 3.0-6.0 | | | F | NADH4 (Mitochondrial gene) | None | 30, 135 |
| BNO93 | 0-0.5 | | | F | SGPL1 (Sphingosine-1-phosphate lyase 1) | Hs 186613 | 31, 136 |
| BNO381 | 0-0.5 | | | F | COBW-like protein | Hs 7535 | 32, 137 |
| BNO96 | 0.5-0 | P | S | B | GNG12 (G-coupled receptor protein γ₁₂ subunit) | Hs 118520 | 33, 138 |
| BNO97⁵ | 0-0.5 | | | A | SDFR1 (Stromal cell-derived factor receptor 1) | Hs 6354 | 34, 139 |
| BNO98 | 0-0.5 | V/F P? | | B | RYBP (Ring1 and YY1 binding protein) | Hs 7910 | 35, 140 |
| BNO99⁵ | 0.5-0 | NR | | C | BMP2 (Bone morphogenic protein 2) | Hs 73853 | 36, 141 |
| BNO101^{5,6} | 3-6, 0-0.5 | | | A | TCEBIL (Transcription elongation factor B (SIII), | Hs 171626 | 37, 142 |
| | | | | | polypeptide 1-like) | | |
| BNO102 | 0-0.5 | | | A | PSME2 (Proteosome activator subunit 2 - | Hs 179774 | 38, 143 |
| | | | | | PA28beta) | | |
| BNO103^{5,6} | 0-0.5, 3.0-6.0, | | | A | FTL (Ferritin, light polypeptide) | Hs 11134 | 39, 144 |
| | 0.5-0 | | | | | | |
| BNO104 | 3-6 | | | A | ITCH (Itchy homolog E3 ubiquitin protein ligase) | Hs 98074 | 40, 145 |
| BNO105 | 3-6 | | | A | ENO1-alpha (Enolase 1 alpha) | Hs 254105 | 41, 146 |
| BNO106 | 0.5-3 | | | A | HNRPH2 (Heterogeneous nuclear | Hs 278857 | 42, 147 |
| | | | | | ribonucleoprotein H2) | | |
| BNO107 | 0-0.5 | | | A | UNR (NRAS-related gene) | Hs 69855 | 43, 148 |
| BNO108^{5,6} | 0-0.5, 0.5-3.0, | | | A (↑24 hr) | COX-1 (Cytochrome oxidase 1 small subunit - | None | 44, 149 |
| | 6.0-24 | | | | Mitochondrial gene) | | |
| BNO111 | 3-6 | | | A/(B/D) | ZFP36L2 (Zinc finger protein 36, C3H type-like 2) | Hs 78909 | 45, 150 |
| BNO112 | 6-24 | | | A (↑24 hr) | CALM1 (Calmodulin-1) | Hs 177656 | 46, 151 |
| BNO114 | 6-24 | | | A | CYPIBI (Cytochrome P450, subfamily I | Hs 154654 | 47, 152 |
| | | | | | (dioxin-inducible), polypeptide 1) | | |
| BNO115 | 3-6 | | | A | UGTREL1 (UDP-galactose transporter related) | Hs 154073 | 48, 153 |
| BNO116 | 0.5-3 | | | A (↑24 hr) | MCPR (Anaphase-promoting complex 1; meiotic | Hs 40137 | 49, 154 |
| | | | | | checkpoint regulator) | | |
| BNO120 | 3-6 | | | B | GLO1 (Glyoxalase 1) | Hs 75207 | 50, 155 |
| BNO122 | 0.5-3 | | | B? | RPL15 (Ribosomal protein L15) | Hs 74267 | 51, 156 |
| BNO123 | 3-6 | | | B | SF3B1 (splicing factor 3b, subunit 1) | Hs 334826 | 52, 157 |
| BNO124 | 0.5-3 | | | B | AKAP12 (A kinase (PRKA) anchor protein | Hs 788 | 53, 158 |
| | | | | | (gravin) 12) | | |
| BNO128 | 6-24 | | | B | HSPA8 (Heat shock 70 kD protein 8) | Hs 180414 | 54, 159 |
| BNO130 | 0.5-3 | | | B | LIPG (Endothelial lipase) | Hs 65370 | 55, 160 |
| BNO131 | 0-0.5 | 3-D | | C | PX19 like (Px19-like protein) | Hs 279529 | 56, 161 |
| BNO132 | 0.5-0 | | | C | PDCD6 (Programmed cell death 6) | Hs 80019 | 57, 162 |
| BNO133 | 0.5-3 | | | C | SDPR (Serum deprivation response - | Hs 26530 | 58, 163 |
| | | | | | phosphatidylserine binding protein) | | |
| BNO134 | 0.5-0 | | | C | GPI (Glucose phosphate isomerase) | Hs 279789 | 59, 164 |
| BNO135 | 0.5-0 | | | C | COX-3 (Cytochrome oxidase 3 subunit - | None | 60, 165 |
| | | | | | Mitochondrial gene) | | |
| BNO137 | 3-6 | 3-D | M | D | PTRF (Polymerase I and transcript release factor) | Hs 29759 | 61, 166 |
| BNO140 | 3-6 | | | D | CCND2 (Cyclin D2) | Hs 75586 | 62, 167 |
| BNO141 | 0.5-0 | | | D | GOLGA2 (Golgi autoantigen, golgin subfamily a, 2) | Hs 24049 | 63, 168 |
| BNO142 | 3-6 | | | E(↑6 hr) | RPL11(Ribosomal protein L11) | Hs 179943 | 64, 169 |
| BNO144⁵ | 0.5-0, 0-0.5 | | | E(↓24 hr) | EEF1A1 (Eukaryotic translation elongation factor | Hs 181165 | 65, 170 |
| | | | | | 1 alpha 1) | | |
| BNO145 | 0.5-3 | | | E (↑6 hr) | ATP1A1 (ATPase, Na+/K+ transporting, | Hs 76549 | 66, 171 |
| | | | | | alpha 1 polypeptide) | | |
| BNO146 | 0.5-3 | | | E (↑6 hr) | TAX1BP1 (Tax1 (human T-cell leukemia virus | Hs 5437 | 67, 172 |
| | | | | | type I) binding protein 1) | | |
| BNO147 | 0.5-3 | | | E? | KARP-1BP3 (Ku86 Autoantigen Related Protein | Hs 25132 | 68, 173 |
| | | | | | binding protein 3) | | |
| BNO148 | 3-6 | | | E (↓24 hr) | RPS6 (Ribosomal protein S6 subunit) | Hs 350166 | 69, 174 |
| BNO149 | 6-24 | | | E (↓6 hr) | MRPL22 (Mitochondrial ribosomal protein L22) | Hs 41007 | 70, 175 |
| BNO150 | 3-6 | | | E (↑6 hr) | BAZ2B (Bromodomain adjacent to zinc finger | Hs 8383 | 71, 176 |
| | | | | | domain, 2B) | | |
| BNO151 | 3-6 | | | E (↑24 hr) | TEGT (Testis enhanced gene transcript - BAX | Hs 74637 | 72, 177 |
| | | | | | inhibitor 1) | | |
| BNO152 | 0-0.5 | | | E (↑6 hr) | TDE1 (Tumor differentially expressed 1) | Hs 272168 | 73, 178 |
| BNO153 | 0-0.5 | | | E (↑24 hr) | RPA2 (Replication protein A2) | Hs 79411 | 74, 179 |
| BNO154 | 0-0.5 | | | E (↓24 hr) | PABPC1 (Poly(A) binding protein, cytoplasmic 1) | Hs 172182 | 75, 180 |
| BNO155 | 0.5-0 | | | E | RPS13 (Ribosomal protein S13) | Hs 165590 | 76, 181 |
| BNO156 | 0.5-0 | | | E | TCP1 (T-complex 1) | Hs 4112 | 77, 182 |
| BNO157 | 0.5-0 | | | E | RNASE1 (Ribonuclease, RNase A family, 1) | Hs 78224 | 78, 183 |
| BNO158 | 0-0.5 | | | L | SNX5 (Sorting nexin 5) | Hs 13794 | 79, 184 |
| BNO159 | 0.5-0 | | | L? | NP220 (NP220 nuclear protein) | Hs 169984 | 80, 185 |
| BNO161 | 0-0.5 | | | | DDX15 (DEAD/H (Asp-Glu-Ala-Asp/His) box | Hs 5683 | 81, 186 |
| | | | | | polypeptide 15) | | |
| BNO162 | 0.5-3 | | | | RPL28 (Ribosomal protein L28) | Hs 4437 | 82, 187 |
| BNO163 | 0.5-3 | | | | UBE2L3 (Ubiquitin-conjugating enzyme E2L3) | Hs 108104 | 83, 188 |
| BNO164 | 3-6 | | | | CYTB (Cytochrome b - Mitochondrial gene) | None | 84, 189 |
| BNO166 | 0-0.5 | | | ? | MPHOSPH6 (M-phase phosphoprotein 6) | Hs 152720 | 85, 190 |
| BNO167⁵ | 0-0.5 | | | | SSBP2 (Single-stranded DNA binding protein 2) | Hs 169833 | 86, 191 |
| BNO168 | 0.5-0 | | | | NADH1 (NADH dehydrogenase subunit 1 - | None | 87, 192 |
| | | | | | Mitochondrial gene) | | |
| BNO169 | 0.5-3 | | | U | PHF3 (PHD finger protein 3) | Hs 78893 | 88, 193 |
| BNO170⁵ | 0-0.5, 0.5-0 | | | U | ICMT (Isoprenylcysteine carboxyl methyltransferase) | Hs 183212 | 89, 194 |
| BNO171 | 0-0.5 | | | L | HCFCI (Host cell factor CI - VP16-accessory protein) | Hs 83634 | 90, 195 |
| BNO173 | 0-0.5 | | | L | QKI7/7B (QKI Homolog of mouse quaking QKI - KH | Hs 15020 | 91, 196 |
| | | | | | domain RNA binding protein) | | |
| BNO175 | 0.5-0 | | | L | S100A13 (S100 calcium binding protein A13) | Hs 14331 | 92, 197 |
| BNO176 | 0.5-0 | | | L? | CGI-99 protein | Hs 110803 | 93, 198 |
| BNO177 | 0-0.5 | | | L? | EXTI (Exostoses (multiple) I) | Hs 184161 | 94, 199 |
| BNO179 | 0.5-0 | | | | TI-227H (Mitochondrial gene) | None | 95 |
| BNO180 | 0.5-0 | | | | KPNA2 (Karyopherin alpha 2 (RAG cohort 1 - | Hs 159557 | 96, 200 |
| | | | | | importin alpha 1) | | |
| BNO181⁵ | 0.5-3 | | | | RPS9 (Ribosomal protein S9) | Hs 180920 | 97, 201 |
| BNO182 | 3-6 | | | | NCBP2 (Nuclear cap binding protein subunit 2) | Hs 240770 | 98, 202 |
| BNO183 | 3-6 | | | | S12 rRNA (Mitochondrial gene) | None | 99 |
| BNO366 | 3-6 | | | F | ATP synthase 6 (Mitochondrial gene) | None | 100, 203 |
| BNO367 | 3-6 | | | F | HSP105 (Heat shock protein 105) | Hs 36927 | 101, 204 |
| BNO368 | 0.5-0 | | | L? | PROXI (Prospero-related homeobox I) | Hs 159437 | 102, 205 |
| BNO369⁵ | 6-24, 0.5-0 | | | C/F? | ACTB (actin, beta) | Hs 288061 | 103, 206 |
| BNO370 | 6-24 | | | F | TMSB4X (Thymosin, beta 4 X chromosome) | Hs 75968 | 104, 207 |
| BNO371⁵ | 3-6, 0.5-0 | | | F | 16S rRNA (Mitochondrial gene) | None | 105 |
| BNO373 | 3-6 | | | F | APLP2 (Amyloid beta (A4) precursor-like protein 2) | Hs 279518 | 106, 208 |
| BNO374 | 0-0.5 | | | F | EPLIN beta (Epithelial protein lost in neoplasm beta) | Hs 10706 | 107, 209 |
| BNO375 | 0-0.5 | | | F | EIF3S9 (Eukaryotic translation initiation factor 3 | Hs 57783 | 108, 210 |
| | | | | | subunit 9) | | |
| BNO376 | 0.5-0 | | | F | PSMC1 (Proteasome 26S subunit, ATPase, 1) | Hs 4745 | 109, 211 |
| BNO377 | 0-0.5 | | | F | TCTA (T-cell leukemia translocation altered gene) | Hs 250894 | 110, 212 |
| BNO378 | 0.5-0 | | | F | NTF2 (Nuclear transport factor 2) | Hs 151734 | 111, 213 |
| BNO379 | 3-6 | | | F | MLC-B (Myosin regulatory light chain) | Hs 180224 | 112, 214 |
| BNO380 | 0-0.5 | | | F | CHRNAI (nicotinic acetylcholine receptor | Hs 2266 | 113, 215 |
| | | | | | alpha 1 subunit) | | |
| BNO382 | 0-0.5 | | | F | MAP1B (Microtubule associated protein 1B) | Hs 103042 | 114, 216 |

Note:
¹The time period in which isolated clones were obtained and the direction of subtraction;
²Response to VEGF (V), bFGF (F), PMA (P) on 2-dimensional collagen gels. Response specific to 3-dimensional (3-D) not 2-dimensional collagen gels. No response (NR).
³Expression in several (S) cell types; expression in many (M) cell types.
⁴Specifically relates to the tube and lumen forming assay on 3-dimensional collagen. Class A: up at 0.5 hours; Class B: up at 3 hours; Class C: down at 0.5 hours; Class D: down at 3 hours; Class E: Other regulation patterns; Class F: not regulated; Class L: Virtual Northern limited to 0 and 0.5 hour time points only, but no regulation.
⁵Multiple cDNA clones were identified for this BNO gene.
⁶The multiple cDNA clones identified for this BNO gene showed regulation of expression at more than one time point of the angiogenesis model.

References

References cited herein are listed on the following pages, and are incorporated herein by this reference.
Altschul, S F. et al. (1997). Nucleic Acids Res. 25: 3389-3402.
Asano, T. et al. (1998). Eur. J. Biochem. 251 : 314-319.
Breaker, R R. and Joyce, G F. (1995). Chem. Biol. 2: 655-600.
Cockerill, G W. et al. (1994). A Survey of Cell Biol. 159: 113-160.
Cole, S P. et al. (1984). Mol. Cell Biol. 62: 109-120.
Cote, R J. et al. (1983). Proc. Natl. Acad. Sci. USA 80: 2026-2030.
Culver, K. (1996). Gene Therapy : A Primer for Physicians. Second Edition. (Mary Ann Liebert).
Folkman, J. and Haudenschild, C. (1980). Nature (Lond.) 288: 551-556.
Friedman, T. (1991). In Therapy for Genetic Diseases. (T Friedman (Ed) Oxford University Press. pp 105-121.
Gamble, J R. et al. (1993). J. Cell Biol. 121: 931-943.
Gamble, J R. et al. (1999). Endothelium 7: 23-34.
Gecz, J. et al. (1997). Genomics 44: 201-213.
Goldman, C K. et al. (1997). Nature Biotechnology 15: 462-466.
Haseloff, J. and Gerlach, W L. (1988). Nature 334: 585-591.
Heller, R A. et al. (1997). Proc. Natl. Acad. Sci. USA 94: 2150-2155.
Huse, W D. et al. (1989). Science 246: 1275-1281.
Kohler, G. and Milstein, C. (1975). Nature 256: 495-497.
Kozbor, D. et al. (1985). L. Immunol. Methods 81:31-42.
Leavesley, D I. et al. (1993). J. Cellular Biol. 121: 163-170.
Litwin, M. et al. (1997). J. Cell Biol. 139: 219-228.
Meyer, G T. et al. (1997). The Anatomical Record 249: 327-340.
Orlandi, R. et al. (1989). Proc. Natl. Acad. Sci. USA 86: 3833-3837.
Rayner, J R. and Gonda, T J. (1994). Mol. Cell Biol. 14: 880-887.
Rickert, R C. et al. (1997). Nucleic Acids Res. 25: 1317-1318.
Ridley, A J. (2001). Trends Cell Biol. 11: 471-477.
Sambrook, J. et al. (1989). Molecular cloning: a laboratory manual. Second Edition. (Cold Spring Harbour Laboratory Press, New York).
Scharf, D. et al. (1994). Results Probl. Cell Differ. 20: 125-162.
Schena, M. et al. (1996). Proc. Natl. Acad. Sci. USA 93: 10614-10619.
Schwenk, F. et al. (1995). Nucleic Acids Res. 23: 5080-5081.
Ueda, H. et al. (1997). J. Cell Sci. 110 : 1503-1511.
Ueda, H. et al. (1999). J. Biol. Chem. 274 : 12124-12128.
Van Aelst, L. and D'Souza-Schorey, C. (1997). Genes Devel. 11: 2295-2322.
Winter, G. et al. (1991). Nature 349: 293-299.
Xia, P. et al. (1999). J. Biological Chem. 274: 34499-34505.
Yasuda, H. et al. (1998). J. Biol. Chem. 273: 21958-21965.

1. An isolated nucleic acid molecule comprising the sequence set forth in one of SEQ ID Numbers: 1 to 20.

2. An isolated nucleic acid molecule comprising the sequence set forth in one of SEQ ID Numbers: 1 to 114, or a fragment thereof, and which encodes a polypeptide that plays a role in an angiogenic process.

3. An isolated nucleic acid molecule that is at least 70% identical to a nucleic acid molecule comprising the sequence set forth in one of SEQ ID Numbers: 1 to 114, and which encodes a polypeptide that plays a role in an angiogenic process.

4. An isolated nucleic acid molecule as claimed in claim 3 that is at least 85% identical.

5. An isolated nucleic acid molecule as claimed in claim 3 that is at least 95% identical.

6. (cancelled).

7. An isolated nucleic acid molecule that encodes a polypeptide that plays a role in an angiogenic process, and which hybridizes under stringent conditions with a nucleic acid molecule comprising the nucleotide sequence set forth in one of SEQ ID Numbers: 1 to 114.

8. (cancelled).

9. (cancelled).

10. An isolated nucleic acid molecule consisting any one of the nucleotide sequences set forth in SEQ ID Numbers: 1 to 20.

11. Use of a nucleic acid molecule selected from the group consisting of DNA molecules having the sequence set forth in SEQ ID Numbers: 70, 72 to 73, 78, 83 to 87, 89, 160 or 174 to identify and/or obtain full-length human genes involved in an angiogenic process.

12. (cancelled).

13. (cancelled).

14. A gene when identified by the use of a DNA molecule selected from any one of SEQ ID Numbers: 70, 72 to 73, 78, 83 to 87, 89, 160 or 174.

15. (cancelled).

16. (cancelled).

17. (cancelled).

18. (cancelled).

19. (cancelled).

20. (cancelled).

21. (cancelled).

22. (cancelled).

23. (cancelled).

24. An expression vector comprising a nucleic acid molecule as claimed in any one of claims 1, 2, 3 or 7.

25. A cell comprising an expression vector as claimed in claim 24.

26. (cancelled).

27. A method of preparing a polypeptide comprising the steps of:
(1) culturing cells as claimed in either one of claims 25 under conditions effective for polypeptide production; and
(2) harvesting the polypeptide.

28. A polypeptide prepared by the method of claim 27 or encoded by a nucleic acid as claimed in any one of claims 1, 2, 3 or 7.

29. A method of modulating angiogenesis comprising modulating the expression or activity of a polypeptide in a cell, wherein the polypeptide is encoded by a nucleic acid sequence as claimed in claim 1 or 2.

30. The method of claim 29 wherein the nucleic acid sequence is selected from the group consisting of SEQ ID Numbers: 1 to 20.

31. (cancelled).

32. (cancelled).

33. The method of claim 29 wherein the polypeptide forms part of a polypeptide complex.

34. The method of claim 29 wherein the expression or activity of the polypeptide is modulated by introducing into the cell an antagonist or agonist of the nucleic acid molecule or a polypeptide.

35. The method of claim 29 wherein the expression or activity of the polypeptide is modulated by introducing into the cell an antisense to an the nucleic acid molecule.

36. The method of claim 29 wherein the expression or activity of the polypeptide is modulated by introducing into the cell a nucleic acid molecule which is the complement of at least a portion of a the nucleic acid sequence and is claimed in any one of claims 1 to 10 and is capable of modulating expression or levels of said nucleic acid sequence.

37. The method of claim 36 wherein the nucleic acid molecule is an RNA molecule that hybridizes with the mRNA encoded by the nucleic acid sequence.

38. The method of claim 36 wherein the nucleic acid molecule is a catalytic nucleic acid molecule that is targeted to the nucleic acid sequence.

39. The method of claim 38 wherein the catalytic nucleic acid molecule is selected from the group consisting of a DNAzyme and a ribozyme.

40. (cancelled).

41. The method of claim 29 wherein the polypeptide expression or activity is modulated by an antibody capable of binding the polypeptide.

42. The method of claim 41 wherein the antibody is a fully human antibody.

43. The method of claim 41 wherein the antibody is selected from the group consisting of a monoclonal antibody, a humanised antibody, a chimaeric antibody or an antibody fragment including a Fab fragment, (Fab′)₂ fragment, Fv fragment, single chain antibodies and single domain antibodies.

44. The method of claim 29 wherein the polypeptide expression or activity is modulated by introducing into the cell a nucleic acid molecule comprising the nucleic acid sequence, or an active fragment or variant thereof.

45. The method of claim 44 wherein the nucleic acid molecule is introduced by way of an expression vector as claimed in claim 24.

46. (cancelled).

47. The method of any one of claims 29 to 46 wherein angiogenesis is uncontrolled or enhanced.

48. The method of any one of claims 29 to 46 wherein angiogenesis is inappropriately arrested or decreased.

49. (cancelled).

50. (cancelled).

51. (cancelled).

52. (cancelled).

53. (cancelled).

54. (cancelled).

55. (cancelled).

56. (cancelled).

57. (cancelled).

58. (cancelled).

59. (cancelled).

60. (cancelled).

61. (cancelled).

62. (cancelled).

63. (cancelled).

64. (cancelled).

65. (cancelled).

66. (cancelled).

67. (cancelled).

68. (cancelled).

69. (cancelled).

70. (cancelled).

71. (cancelled).

72. (cancelled).

73. (cancelled).

74. (cancelled).

75. (cancelled).

76. (cancelled).

77. (cancelled).

78. (cancelled).

79. (cancelled).

80. (cancelled).

81. (cancelled).

82. (cancelled).

83. (cancelled).

84. (cancelled).

85. (cancelled).

86. (cancelled).

87. (cancelled).

88. (cancelled).

89. (cancelled).

90. (cancelled).

91. (cancelled).

92. (cancelled).

93. The use of a nucleic acid molecule as claimed in any one of claims 1, 2, 3 or 7 for the screening of candidate pharmaceutical compounds useful in the treatment of angiogenesis-related disorders.

94. A compound useful in the treatment of angiogenesis-related disorders when identified by the use of a nucleic acid molecule as claimed in any one of claims 1, 2, 3 or 7.

95. The use of a polypeptide as claimed in claim 28, or a polypeptide complex as claimed in claim 23 for the screening of candidate pharmaceutical compounds useful in the treatment of angiogenesis-related disorders.

96. A compound useful in the treatment of angiogenesis-related disorders when identified by the use of a polypeptide as claimed in claim 28, or a polypeptide complex as claimed in claim 23.

97. The use of a cell as claimed in claim 25 for the screening of candidate pharmaceutical compounds useful in the treatment of angiogenesis-related disorders.

98. A compound useful in the treatment of angiogenesis-related disorders when identified by the use of a cell as claimed in claim 25.

99. A method of screening for a candidate pharmaceutical compound useful in the treatment of angiogenesis-related disorders comprising the steps of:
(1) providing a polypeptide as claimed in claim 28, or a polypeptide complex as claimed in claim 23;
(2) adding a candidate pharmaceutical compound to said polypeptide; and
(3) determining the binding of said candidate compound to said polypeptide;
wherein a compound that binds to the polypeptide or polypeptide complex is a candidate pharmaceutical compound.

100. A method of screening for candidate pharmaceutical compound useful in the treatment of angiogenesis-related disorders comprising the steps of:
(1) providing a cell, as claimed in claim 25;
(2) adding a candidate pharmaceutical compound to said cell; and
(3) determining the effect of said candidate pharmaceutical compound on the functional properties of said cell;
wherein a compound that alters the functional properties of said cell is a candidate pharmaceutical compound.

101. A method of screening for a candidate pharmaceutical compound useful in the treatment of angiogenesis-related disorders comprising the steps of:
(1) providing a cell, as claimed in claim 25;
(2) adding a candidate pharmaceutical compound to said cell; and
(3) determining the effect of said candidate pharmaceutical compound on the expression of the nucleic acid molecule that is part of the expression vector in said cell;
wherein a compound that alters the expression of the nucleic acid molecule that is part of the expression vector in said cell is a candidate pharmaceutical compound.

102. A method of screening for a candidate pharmaceutical compound useful in the treatment of angiogenesis-related disorders comprising the steps of:
(1) providing a cell, as claimed in claim 25;
(2) adding a candidate pharmaceutical compound to said cell; and
(3) determining the effect of said candidate pharmaceutical compound on the expression or activity of the polypeptide encoded by the nucleic acid molecule that is part of the expression vector in said cell;
wherein a compound that alters the expression or activity of polypeptide encoded by the nucleic acid molecule that is part of the expression vector in said cell is a candidate pharmaceutical compound.

103. A compound when identified by the method of any one of claims 100 to 102.

104. A pharmaceutical composition comprising a compound as claimed in 103 and a pharmaceutically acceptable carrier.

105. An antibody which is immunologically reactive with an isolated polypeptide as claimed in claim 15.

106. An antibody as claimed in claim 105 which is a fully human antibody.

107. An antibody as claimed in claim 105 which is selected from the group consisting of a monoclonal antibody, a humanised antibody, a chimaeric antibody or an antibody fragment including a Fab fragment, (Fab′)₂ fragment, Fv fragment, single chain antibodies and single domain antibodies.

108. A catalytic nucleic acid targeted to a nucleic acid sequence as claimed in claim 1.

109. A catalytic nucleic acid of claim 108 which is a DNAzyme.

110. A catalytic nucleic acid of claim 108 which is a ribozyme.

111. Use of a nucleic acid molecule as claimed in any one of claims 1, 2, 3 or 7 in the diagnosis or prognosis of an angiogenesis-related disorder.

112. Use of a polypeptide as claimed in claim 28 in the diagnosis or prognosis of an angiogenesis-related disorder.

113. Use of an antibody as claimed in claim 28 or an antibody to a polypeptide as claimed in claim 16 in the diagnosis or prognosis of an angiogenesis-related disorder.

114. A method for the diagnosis or prognosis of an angiogenesis-related disorder comprising the steps of:
(1) establishing a profile for normal expression and/or activity of a gene as comprising a nucleic acid as claimed in claims 1 or 2 in unaffected subjects;
(2) measuring the level of expression and/or activity of the gene in a person suspected of abnormal expression and/or activity of the gene; and
(3) comparing the measured level of expression and/or activity with the profile for normal expression and/or activity;
wherein an altered level of expression and/or activity in said subject is an indication of an angiogenesis-related disorder, or a predisposition thereto.

115. A method as claimed in claim 114 wherein reverse transcriptase PCR is employed to measure levels of expression.

116. A method as claimed in claim 114 wherein a hybridisation assay using a probe derived from the gene, or a fragment thereof, is employed to measure levels of expression.

117. A method for the diagnosis or prognosis of an angiogenesis-related comprising the steps of:
(1) obtaining DNA from a subject corresponding to a nucleic acid sequence as claimed in claims 1 or 2; and
(2) comparing the DNA from said to the DNA of the corresponding wild-type gene;
wherein altered DNA properties in said subject is an indication of an angiogenesis-related disorder, or a predisposition thereto.

118. A method as claimed in claim 117 wherein the DNA of the gene is sequenced and the sequences compared.

119. A method as claimed in claim 117 wherein the DNA of the gene is subjected to SSCP analysis.

120. A method for the diagnosis or prognosis of an angiogenesis-related disorder comprising the steps of:
(1) establishing a physical property of a wild-type polypeptide as claimed in claim 28;
(2) obtaining the polypeptide from a person suspected of an abnormality of that polypeptide; and;
(3) measuring the property for the polypeptide expressed by the person and comparing it to the established property for wild-type polypeptide;
wherein altered polypeptide properties in said subject is an indication of an angiogenesis-related disorder, or a predisposition thereto.

121. A method as claimed in claim 120 wherein the property is the electrophoretic mobility.

122. A method as claimed in claim 120 wherein the property is the proteolytic cleavage pattern.

123. A genetically modified non-human animal comprising a nucleic acid molecule as claimed in any one of claims 1, 2, 3 or 7 or in which a nucleic acid as claimed in claim 1 or 2 is disrupted.

124. A genetically modified non-human animal as claimed in claim 123 in which the animal is selected from the group consisting of rats, mice, hamsters, guinea pigs, rabbits, dogs, cats, goats, sheep, pigs and non-human primates such as monkeys and chimpanzees.

125. A genetically modified non-human animal as claimed in claim 123 wherein the animal is a mouse.

126. Use of a genetically modified non-human animal as defined claim 123 in screening for candidate pharmaceutical compounds useful for the treatment of angiogenesis-related disorders.

127. (cancelled).

128. (cancelled).

129. (cancelled).

130. (cancelled).
